(19) United States
(12) Patent Application Publication
(10) Pub. No.: US 2026/0241143 A1
(43) Pub. Date: Aug. 20, 2026

(54) WIRE TERMINATION OF STEERABLE CATHETER

(71) Applicant: Auris Health, Inc., Santa Clara, CA (US)

(72) Inventors: Justin GILBERT, Santa Clara, CA (US); Joseph SHING, Cupertino, CA (US)

(73) Assignee: Auris Health, Inc., Santa Clara, CA (US)

(21) Appl. No.: 19/057,288

(22) Filed: Feb. 19, 2025

(51) Int. Cl. A61M 25/01; A61M 25/00

(52) U.S. Cl. CPC A61M 25/0147; A61M 25/001; A61M 2025/015; A61M 2207/00

(57) ABSTRACT

A steerable catheter includes an elongate shaft having opposing distal and proximal ends, a wire extending within a wall of the elongate shaft between the distal and proximal ends, and a distal tip arranged at the distal end. The distal tip including an annular body, and a wire channel defined in the annular body and providing a path sized to receive the wire and including first and second axial legs and a transverse portion interconnecting the first and second axial legs. The wire is routed through the wire channel such that opposing ends of the wire extend proximally from the distal top.

### BACKGROUND

Various medical procedures involve the use of one or more medical devices for accessing a target anatomical site in a patient. In some instances, the improper use of certain devices when accessing the site in connection with a procedure can adversely affect the health of the patient, the integrity of the medical device(s), and/or the efficacy of the procedure.

Some medical devices include a steerable catheter used to access various internal parts and organs of a patent and provide a working channel for tools to access such parts and organs. Steerable catheters commonly contain wires that can be manipulated (e.g., pulled) to help steer the catheter. These wires typically terminate at the distal end of the catheter to transfer the force of pulling to articulate the catheter. The wires are small and difficult to secure, given the high forces they are exposed to. Laser welding and soldering the wires to the distal end of the catheter can sensitize the wire material and otherwise degrade its mechanical properties.

### SUMMARY OF THE INVENTION

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to embodiments of the present disclosure, a steerable catheter includes an elongate shaft having opposing distal and proximal ends, a wire extending within a wall of the elongate shaft between the distal and proximal ends, and a distal tip arranged at the distal end and including, the distal tip including an annular body and a wire channel defined in the annular body and providing a path sized to receive the wire and including first and second axial legs and a transverse portion interconnecting the first and second axial legs, wherein the wire is routed through the wire channel such that opposing ends of the wire extend proximally from the distal tip.

In accordance with other embodiments, a distal tip for a steerable catheter includes an annular body, the annual body providing opposing first and second ends, a first portion provided at the first end, and a second portion contiguous with and extending proximally from the first portion. The distal tip also includes a wire channel defined in the second portion in the form of a path and sized to receive a wire such that opposing ends of the wire extend proximally from the annular body, wherein a diameter of the first portion is larger than a diameter of the second portion such that a first shoulder is provided at an interface between the first and second portions.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 illustrates an example robotic medical system arranged for a diagnostic and/or therapeutic ureteroscopy procedure in accordance with one or more embodiments.

FIG. 2 illustrates an example robotic medical system arranged for a diagnostic and/or therapeutic bronchoscopy procedure in accordance with one or more embodiments.

FIG. 3 illustrates an example table-based robotic system in accordance with one or more embodiments.

FIG. 4 illustrates example medical system components that may be implemented in any of the medical systems of FIGS. 1-3 in accordance with one or more embodiments.

FIG. 5 illustrates an example catheter disposed in the kidney of a patient in accordance with one or more embodiments.

FIG. 6 illustrates an example catheter including a shaft and a handle in accordance with one or more embodiments.

FIG. 7A illustrates a side view of the shaft of the catheter from FIG. 6 in accordance with one or more embodiments.

FIG. 7B illustrates a cross-sectional view of the shaft of the catheter from FIG. 6 in accordance with one or more embodiments.

FIG. 8 illustrates a perspective view of the shaft of the catheter from FIG. 6 in accordance with one or more embodiments.

FIGS. 9A and 9B are enlarged isometric and side views, respectively, of a distal tip of a catheter, according to one or more embodiments.

FIGS. 9C and 9D are isometric back and front views, respectively, of the distal tip of FIGS. 9A-9B, according to one or more additional embodiments.

FIGS. 10A and 10B are enlarged isometric and side views, respectively, of another example distal tip of a catheter, according to one or more additional embodiments.

FIG. 10C is an axial end view of the distal tip of FIGS. 10A-10B showing at least one embodiment of an overhang entrapment feature, according to one or more embodiments.

FIG. 11 is a schematic flowchart depicting an example method for assembling a distal tip of a steerable catheter, in accordance with the principles of the present disclosure.

### DETAILED DESCRIPTION

The present disclosure is related to steerable catheters and, more particularly, to distal tips for steerable catheters that include wire channels for securing wires.

Embodiments disclosed herein describe embodiments of a steerable catheter that includes an elongate shaft having opposing distal and proximal ends, at least one wire extending within a wall of the elongate shaft between the distal and proximal ends, and a distal tip arranged at the distal end of the shaft. The distal tip includes an annular body, and a wire channel defined in the body and providing a path including first and second axial legs and a transverse portion interconnecting the first and second axial legs. The wire is routed through the wire channel such that opposing ends of the wire extend proximally from the distal tip. The wire can be mechanically attached to the distal tip in a manner that does not impact the material properties of the wire. In some embodiments, for example, a ring may be arranged over the interface between the wire and the wire channel and may be secured to the distal tip. In applications where the ring is welded to the distal tip, the welding operation is undertaken such that the amount of heat introduced to the wire is limited or entirely avoided.

Although certain aspects of the present disclosure are described in detail herein in the context of renal, urological, and/or nephrological procedures, such as kidney stone removal/treatment procedures, it should be understood that such context is provided for convenience, and the concepts disclosed herein are applicable to any suitable medical procedures, such as a bronchoscopy. The following description is also applicable to other surgical/medical operations or medical procedures concerned with the removal of objects from a patient, including any object that can be removed from a treatment site or patient cavity (e.g., the esophagus, ureter, intestine, eye, etc.) via percutaneous and/or endoscopic access, such as, for example, gallbladder stone removal, lung (pulmonary/transthoracic) tumor biopsy, cataract removal, etc. However, as mentioned, description of the renal/urinary anatomy and associated medical issues and procedures is presented below to aid in the description of the concepts disclosed herein.

Several methods can be used for treating patients with kidney stones, including observation, medical treatments (such as expulsion therapy), non-invasive treatments (such as extracorporeal shock wave lithotripsy (ESWL)), minimally-invasive or surgical treatments (such as ureteroscopy and percutaneous nephrolithotomy (“PCNL”)), and so on. In some approaches (e.g., ureteroscopy and PCNL), the physician gains access to the stone, the stone is broken into smaller pieces or fragments, and the relatively small stone fragments/particulates are extracted from the kidney using a basketing device and/or aspiration.

In ureteroscopy procedures, a physician may insert a ureteroscope into the urinary tract through the urethra to remove urinary stones from the bladder and ureter. A ureteroscope can include an imaging device at its distal end configured to enable visualization of the urinary tract. The ureteroscope can also include a lithotripsy device to capture or break apart urinary stones. During a ureteroscopy procedure, one physician/technician may control the position of the ureteroscope, while another physician/technician may control the lithotripsy device(s).

In PCNL procedures, which may be used to remove relatively large stones, a physician may insert a nephroscope through the skin (i.e., percutaneously) and intervening tissue to provide access to the treatment site for breaking-up and/or removing the stone(s). During PCNL procedures, fluidics can be applied to clear stone dust, small fragments, and/or thrombus from the treatment site and/or the visual field. In some instances, a relatively straight and/or rigid nephroscope is used, wherein the physician positions the tip of the nephroscope at the appropriate location within the kidney (e.g., calyx) by pushing/leveraging the device against the patient's body. This movement can be harmful to the patient (e.g., cause tissue damage).

In other procedures, such as one or more of those discussed in further detail below, a physician can use multiple instruments via a percutaneous and/or direct access path to remove a kidney stone. For example, a physician can navigate a scope to a target site in a kidney through the urethra in a patient and insert a catheter device into the target site through the skin of the patient. The physician can use the scope and the catheter device in cooperation to fragment the kidney stone and extract the fragments from the patient.

The present disclosure relates to systems, devices, and methods for navigating to and/or aspirating/irrigating a target site to perform a medical procedure. For example, a catheter can be implemented that includes an elongate shaft and a handle/base coupled to the shaft and configured to control actuation of the shaft (at least at a distal portion of the shaft). The shaft can include a lumen configured to couple to an aspiration/irrigation system to provide aspiration/irrigation to a target site, such as to remove an object from a patient. The handle/base of the catheter can be controlled robotically and/or manually to articulate the distal portion of the shaft, so that the catheter can be navigated within the anatomy of a patient. For instance, the catheter can include multiple pull wires or other elongate movement members that are coupled to the distal portion (end) of the shaft and one or more manipulation components in the handle of the catheter. The pull wires/elongate movement members can be manipulated (using the handle) to control movement of the distal portion of the shaft. Additionally, or alternatively, the handle of the catheter can be moved to control movement of the distal portion of the catheter, such as to insert/retract/roll the tip of the catheter.

In some embodiments, the techniques and devices discussed herein can enable objects to be removed from patients in an efficient manner that prevents damage to the anatomy of the patients and/or damage to the removal devices. For example, the articulable catheter structures discussed herein can enable a physician to navigate a distal portion of a catheter within a patient without moving an entirety of the catheter (e.g., by controlling one or more elements within a handle/base of the catheter). In contrast, some nephoscopy procedures require a physician to leverage a proximal portion of a nephroscope to place a tip of the nephroscope in the appropriate location within the patient, resulting in damage to the anatomy of the patient.

In some implementations, the techniques discussed herein implement robotic-assisted medical procedures, wherein robotic tools enable a physician to perform endoscopic and/or percutaneous access and/or treatment for a target anatomical site. For example, the robotic tools can engage with and/or control one or more medical instruments, such as a scope, catheter, or another instrument, to access a target site in a patient and/or perform a treatment at the target site. In some cases, the robotic tools are guided/controlled by a physician. In other cases, the robotic tools operate in an automatic or semi-automatic manner. Although some techniques are discussed in the context of robotic-assisted medical procedures, the techniques may be applicable to other types of medical procedures, such as procedures that do not implement robotic tools or implement robotic tools for relatively few operations (e.g., less than a threshold number). For example, the techniques can be applicable to procedures in which a manually operated medical instrument is implemented, such as a manual catheter and/or scope controlled entirely by a physician.

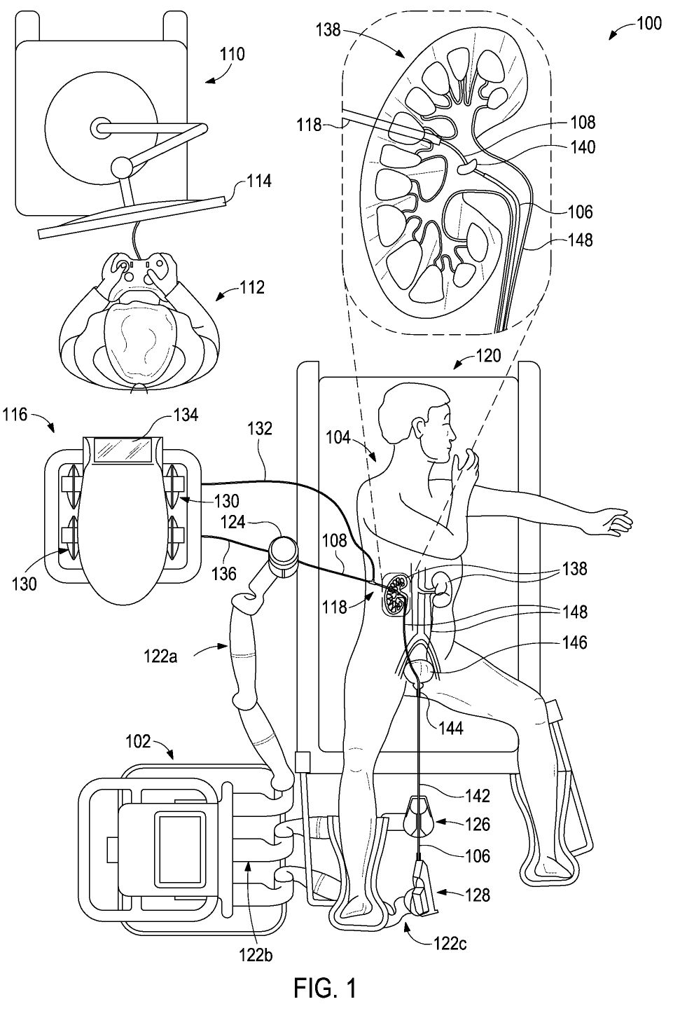

FIG. 1 illustrates an example robotic medical system 100 arranged for a diagnostic and/or therapeutic ureteroscopy procedure. The medical system 100 includes a robotic system 102 configured to engage with and/or control one or more medical instruments/devices to perform a procedure on a patient 104. In the example of FIG. 1, the robotic system 102 is operatively coupled to and drives a scope 106 and a catheter 108. However, the robotic system 102 can couple to or drive other types of medical instruments.

The medical system 100 also includes a control system 110 configured to interface with the robotic system 102 and/or a physician 112, provide information regarding the procedure, and/or perform a variety of other operations. For example, the control system 110 can include one or more display(s) 114 configured to present certain information to assist the physician 112 in performing the procedure.

The medical system 100 can also include a fluid management system 116 (sometimes referred to as an “aspiration system 116” or an “irrigation system 116”) configured to provide aspiration and/or irrigation to a target site, such as via the catheter 108, the scope 106, an instrument/device 118, and/or another instrument/device. The medical system 100 can include a table 120 (e.g., bed) to hold the patient 104.

Various acts are described herein as being performed by the physician 112. These acts can be performed directly by the physician 112, a user under the direction of the physician 112, another user (e.g., a technician), a combination thereof, and/or any other user. The devices/components of the medical system 100 can be arranged in a variety of ways depending on the type procedure, phase of the procedure, user preferences, and so on. For example, the devices/components of the medical system 100 can be arranged in to perform laboratory procedures on non-living cadavers/models/animals for educational or research and development activities.

The control system 110 can generally operate in cooperation with the robotic system 102 to perform the medical procedure. For example, the control system 110 can communicate with the robotic system 102 via a wireless or wired connection to control a medical instrument connected to the robotic system 102, receive an image(s) captured by a medical instrument, and so on. For example, the control system 110 can receive image data from the scope 106 (e.g., an imaging device associated with the scope 106) and display the image data (and/or representations generated therefrom) to the physician 112 to assist the physician 112 in navigating the scope 106 and/or the catheter 108 within the patient 104. The physician 112 can provide input via an input/output (I/O) device, such as a controller, and the control system 110 can send control signals to the robotic system 102 to control movement of the scope 106/catheter 108 connected to the robotic system 102. The scope 106/catheter 108 (and/or another medical instrument) can be configured to move in a variety of manners, such as to articulate, roll, and so on.

The robotic system 102 includes one or more robotic arms configured to engage with and/or control a medical instrument(s)/device. In the illustrated embodiment, the robotic system 102 includes first, second, and third robotic arms 122a, 122b, and 122c. Each robotic arm 122a-c can include multiple arm segments coupled to joints, which can provide multiple degrees of movement. The distal end of each robotic arm 122a-c, referred to herein as an “end effector,” can be configured to couple to an instrument/device. In the example of FIG. 1, the first robotic arm 122a is coupled to a handle 124 of the catheter 108. The second robotic arm 122b is coupled to a scope-driver instrument coupling/device 126, which can facilitate robotic control/advancement of the scope 106. Further, the third robotic arm 122c is coupled to a handle 128 of the scope 106, which can be configured to facilitate advancement and/or operation of the scope 106 and/or a medical instrument that can be deployed through the scope 106, such as an instrument deployed through a working channel of the scope 106. In this example, the second robotic arm 122b and/or the third robotic arm 122c can control movement of the scope 106 (e.g., articulation, roll, etc.). Although three robotic arms 122a-c are connected to particular medical instruments in FIG. 1, the robotic system 102 can include any number of robotic arms that are configured to connect to any type of medical instrument/device.

The fluid management system 116 can be configured to provide and control aspiration and/or irrigation to a target site. As shown, the fluid management system 116 can be configured to hold one or more fluid bags or containers 130 (e.g., IV bags) and/or control fluid flow thereto/therefrom. For example, an irrigation line 132 may be fluidly coupled to one or more of the containers 130 and to an irrigation port of a percutaneous-access device/assembly 118. Irrigation fluid may be provided to the target anatomy via the irrigation line 132 and the percutaneous-access device/assembly 118.

The fluid management system 116 may include certain electronic components, such as a display 134, flow control mechanics, and/or certain associated control circuitry. The fluid management system 116 may comprise a stand-alone tower/cart and may have one or more containers 130 hanging on one or more sides thereof. The system 116 may include a pump with which aspiration fluid may be pulled into a collection container/cartridge via an aspiration channel or tube 136. The aspiration tube 136 may be coupled to the catheter handle 124 to facilitate aspiration via a lumen in the catheter 108.

In the illustrated system 100, the percutaneous-access device 118 is implemented to provide percutaneous access to a kidney 138 of the patient 104. Although various examples are discussed in the context of providing irrigation/aspiration via the catheter 108 and/or the percutaneous-access device/assembly 118, irrigation fluid and/or aspiration may be provided to the treatment site (e.g., kidney) through another device, such as the scope 106, in some cases.

A medical instrument can include a variety of types of instruments, such as a scope (sometimes referred to as an “endoscope”), a catheter, a needle, a guidewire, a lithotripter, a basket retrieval device, forceps, a vacuum, a needle, a scalpel, an imaging probe, an imaging device, jaws, scissors, graspers, needle holder, micro dissector, staple applier, tacker, suction/irrigation tool, clip applier, and so on. A medical instrument can include a direct entry instrument, percutaneous entry instrument, and/or another type of instrument. The medical instrument may be a steerable or non-steerable device. In some embodiments, a surgical tool refers to a device that is configured to puncture or to be inserted through the human anatomy, such as a needle, a scalpel, a guidewire, and so on. However, a surgical tool can refer to other types of medical instruments.

The term “scope” or “endoscope” can refer to any type of elongate medical instrument having image generating, viewing, and/or capturing functionality (or configured to provide such functionality with an imaging device deployed though a working channel) and configured to be introduced into any type of organ, cavity, lumen, chamber, and/or space of a body. For example, a scope or endoscope, such as the scope 106, can refer to a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), a borescope, and so on. A scope/endoscope, in some instances, may comprise a rigid or flexible tube and/or may be dimensioned to be passed within an outer sheath, catheter, introducer, or other lumen-type device, or may be used without such devices. In some embodiments, a scope includes one or more working channels through which additional tools/medical instruments, such as lithotripters, basketing devices, forceps, laser devices, imaging devices, etc., can be introduced into a treatment site.

In one illustrative and non-limiting procedure, the medical system 100 can be used to remove a kidney stone 140 from the patient 104. During setup for the procedure, the physician 112 can position the robotic arms 122 of the robotic system 102 in the desired configuration and/or attach the appropriate medical instruments. For example, the physician 112 can position the first robotic arm 122a near a treatment site and attach an EM field generator (not illustrated), which can assist in tracking a location of the scope 106 and/or other instruments/devices during the procedure. Further, the physician 112 can position the second robotic arm 122b between the legs of the patient 104 and attach the scope-driver instrument coupling 126, which can facilitate robotic control/advancement of the scope 106. In some instances, the physician 112 can insert a sheath/access instrument 142 into the urethra 144 of the patient 104 and/or through the bladder 146 and up the ureter 148. The physician 112 can connect the sheath/access instrument 142 to the scope-drive instrument coupling 126. The sheath/access instrument 142 can include a lumen-type device configured to receive the scope 106, thereby assisting in inserting the scope 106 into the anatomy of the patient 104. However, in some embodiments the sheath/access instrument 142 is not used (e.g., the scope 106 is inserted directly into the urethra 144). The physician 112 can then insert the scope 106 into the sheath/access 142 instrument manually, robotically, or a combination thereof. The physician 112 can attach the handle 128 of the scope 106 to the third robotic arm 122c, which can be configured to facilitate advancement and/or operation of a basketing device, laser device, and/or another medical instrument deployed through the scope 106.

The physician 112 can interact with the control system 110 to cause the robotic system 102 to advance and/or navigate the scope 106 into the kidney 138. For example, the physician 112 can navigate the scope 106 using a controller or other I/O device to locate the kidney stone 140. The control system 110 can provide information via the display(s) 114 regarding the scope 106 to assist the physician 112 in navigating the scope 106, such as to view an image representation (e.g., a real-time image(s) captured by the scope 106). In some embodiments, the control system 110 can use localization techniques to determine a position and/or an orientation of the scope 106, which can be viewed by the physician 112 through the display(s) 114, in some cases. Further, other types of information can also be presented through the display(s) 114 to assist the physician 112 in controlling the scope 106, such as x-ray images of the internal anatomy of the patient 104.

Once at the site of the kidney stone 140 (e.g., within the calyx of the kidney 138), the scope 106 can be used to designate/tag a target location for a catheter to access the kidney 138 percutaneously. To minimize damage to the kidney 138 and/or the surrounding anatomy, the physician 112 can designate a papilla as the target location for entering into the kidney 138 percutaneously. However, other target locations can be designated or determined. In some embodiments of designating the papilla, the physician 112 can navigate the scope 106 to contact the papilla, the control system 110 can use localization techniques to determine a location of the scope 106 (e.g., a location of the distal end of the scope 106), and the control system 110 can associate the location of the scope 106 with the target location. Further, in some embodiments, the physician 112 can navigate the scope 106 to be within a particular distance to the papilla (e.g., park in front of the papilla) and provide input indicating that the target location is within a field-of-view of the scope 106. The control system 110 can perform image analysis and/or other localization techniques to determine a location of the target location. Moreover, in some embodiments, the scope 106 can deliver a fiduciary to mark the papilla as the target location.

When the target location is designated, the catheter 108 can be inserted through a percutaneous access path into the patient 104 to reach the target site (e.g., rendezvous with the scope 106). For example, the catheter 108 can be connected to the first robotic arm 122a (upon removing the EM field generator) and the physician 112 can interact with the control system 110 to cause the robotic system 102 to advance and/or navigate the catheter 108, as shown in FIG. 1. Alternatively, or additionally, the catheter 108 can be manually inserted and/or controlled, such as when the catheter 108 is implemented as a manually-controllable catheter. In some embodiments, a needle or another medical instrument is inserted into the patient 104 to create the percutaneous access path. The control system 110 can provide information via the display(s) 114 regarding the catheter 108 to assist the physician 112 in navigating the catheter. For example, the display(s) 114 can provide image data from the perspective of the scope 106, wherein the image data may depict the catheter 108 (e.g., when within the field-of-view of an imaging device).

With the scope 106 and/or the catheter 108 located at the target location, the physician 112 can use the scope 106 to break up the kidney stone 140 and/or use the catheter 108 to extract pieces of the kidney stone 140 from the patient 104. For example, the scope 106 can deploy a tool (e.g., a laser, a cutting instrument, lithotripter, etc.) through a working channel to fragment the kidney stone 140 into pieces and the catheter 108 can suck out the pieces from the kidney 138 through the percutaneous access path. The catheter 108 can provide aspiration to maintain/hold the kidney stone 140 at a distal end of the catheter 108 and/or at a relatively fixed position, while the scope 106 fragments the kidney stone 140 using a tool (e.g., laser), as shown in FIG. 1. The fluid management system 116 can provide irrigation to the target site via the percutaneous-access device/assembly 118 and/or provide aspiration to the target site via the catheter 108 (e.g., a lumen in the catheter 108).

Although various example procedures are discussed in the context of implementing a robotically controlled catheter 108, the procedure can be implemented with a manually controllable catheter. For example, the catheter 108 can include a manually controllable handle that is configured to be held/manipulated by the physician 112. The physician 112 can navigate the catheter 108 by rolling, inserting, retracting, or otherwise manipulating the handle and/or a manual actuator, which can result in articulation of a distal portion of the catheter 108. Example robotically controllable and manually controllable catheters are discussed in further detail below.

Although various techniques/systems are discussed as being implemented as robotically assisted procedures (e.g., procedures that at least partly use the medical system 100), the techniques/systems can be implemented in other procedures, such as in fully robotic medical procedures, human-only procedures (e.g., free of robotic systems), and so on. For example, the medical system 100 can be used to perform a procedure without a physician holding/manipulating a medical instrument and without a physician controlling movement of a robotic system/arm (e.g., a fully robotic procedure that relies on relatively little input to direct the procedure). That is, medical instruments that are used during a procedure can each be held/controlled by components of the medical system 100, such as the robotic arms 122 of the robotic system 102.

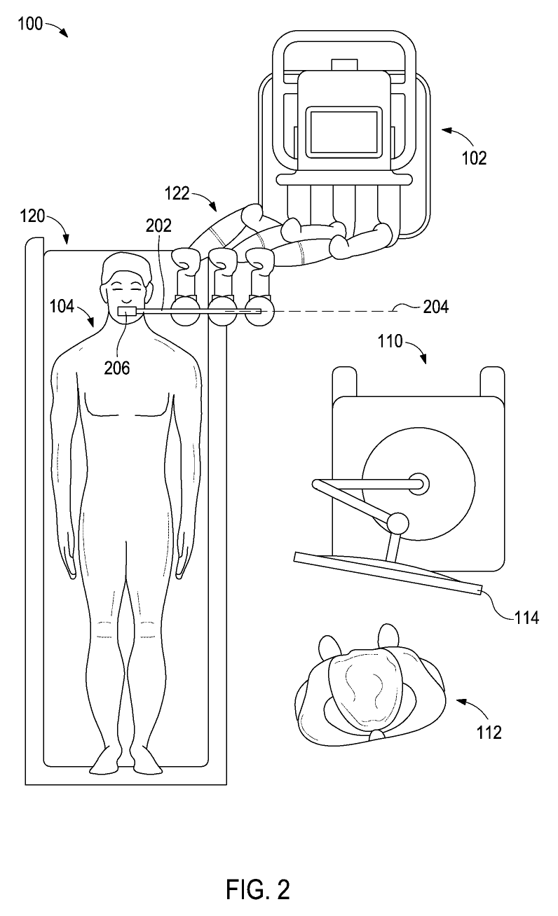

FIG. 2 illustrates the example robotic medical system 100 arranged for a diagnostic and/or therapeutic bronchoscopy procedure in accordance with one or more embodiments. During a bronchoscopy, the arm(s) 122 of the robotic system 102 may be configured to deliver a medical instrument, such as a steerable endoscope 202, which may be a procedure-specific bronchoscope for bronchoscopy, to a natural orifice access point (i.e., the mouth of the patient 104 positioned on the table 120 in the present example) to deliver diagnostic and/or therapeutic tools. As shown, the robotic system 102 (e.g., cart) may be positioned proximate to the patient's upper torso in order to provide access to the access point. Similarly, the robotic arms 122 may be actuated to position the endoscope 202 relative to the access point. The arrangement in FIG. 2 may also be utilized when performing a gastro-intestinal (GI) procedure with a gastroscope, a specialized endoscope for GI procedures.

Once the robotic system 102 is properly positioned, the robotic arms 122 may insert the steerable endoscope 202 into the patient robotically, manually, or a combination thereof. The steerable endoscope 202 may comprise at least two telescoping parts, such as an inner leader portion and an outer sheath portion, with each portion coupled to a separate instrument driver from a set of instrument drivers and/or with each instrument driver coupled to the distal end of a respective robotic arm 122. This linear arrangement of the instrument drivers creates a “virtual rail” 204 that may be repositioned in space by manipulating the one or more robotic arms 122 into different angles and/or positions. The virtual rails/paths described herein are depicted in the figures using dashed lines that generally do not depict any physical structure of the system. Translation of one or more of the instrument drivers along the virtual rail 204 can advance or retract the endoscope 202 from the patient 104.

The endoscope 202 may be directed down the patient's trachea and lungs after insertion using precise commands from the robotic system 102 until reaching the target operative site. In the arrangement of the system 100 in FIG. 2, a patient introducer 206 is attached to the patient 104 via a port (not shown; e.g., surgical tube). The patient introducer 206 may be secured to the table 120 (e.g., via a patient introducer holder configured to support the introducer 206 and secure the position of the patient introducer 206 with respect to the table 120 or other structure). In some embodiments, the patient introducer 206 may include a proximal end, a distal end, and an introducer tube therebetween. The proximal end of the patient introducer 206 can provide an opening/orifice which may be configured to receive the instrument 202 (e.g., bronchoscope), and the distal end of the patient introducer 206 can provide a second opening which may be configured to guide the instrument 202 into the patient-access port. A curved tube component of the introducer 206 can connect the proximal and distal ends thereof and guide the instrument 202 through the introducer 206.

In some embodiments, one or more of the catheters discussed herein can be implemented in a bronchoscopy procedure, such as that illustrated in FIG. 2. For example, a catheter can be implemented in cooperation with or instead of the endoscope 202 to remove an object from the patient 104. In one illustration, a catheter and the endoscope 202 are interchanged on the robotic arms 122 and separately used to investigate/treat a target site. Here, the catheter can be inserted through the patient introducer 206 and used to provide aspiration/irrigation, such as to remove an object from the patient 104. In another illustration, a catheter is deployed through a working channel on the endoscope 202 to provide irrigation/aspiration.

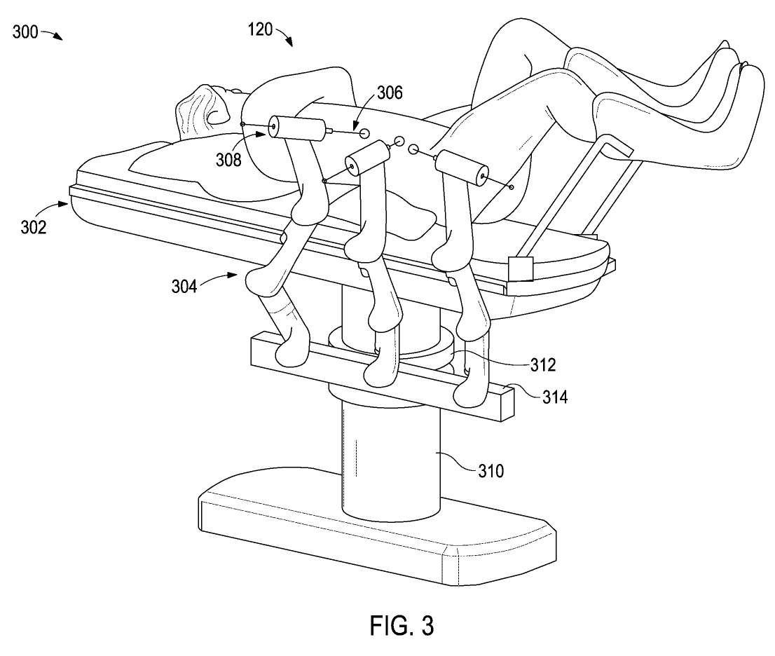

FIG. 3 illustrates a table-based robotic system 300 configured to perform a medical procedure in accordance with one or more embodiments. Here, one or more of the robotic components of the robotic medical system 100 (FIG. 1) can be incorporated into a table 302, which can reduce the amount of capital equipment within an operating room and/or allow greater access to the patient 104 (FIG. 1), in comparison to cart-based robotic systems. For example, the system 300 can include one or more components of the control system 110 (FIG. 1), the robotic system 102 (FIG. 1), and/or the fluid management system 116 (FIG. 1).

As shown, the table 302 can include/incorporate one or more robotic arms 304 (three shown), each configured to engage with and/or control a medical instrument(s)/device 306. Each robotic arm 304 can include multiple arm segments coupled to joints, which can provide multiple degrees of movement. A distal end of each robotic arm 304 (i.e., end effector 308) can be configured to couple to the instrument/device 306, which can include any of the medical instruments/devices discussed herein, such as a catheter, needle, scope, etc. Each robotic arm 304 can be similar to or different than the robotic arms 122 of the system 100 of FIGS. 1 and 2. Further, each end effector 308 can be similar to or different than an end effector of the robotic system 100 of FIG. 1.

As shown, the robotic-enabled table system 300 can include a column 310 coupled to one or more carriages 312 (e.g., ring-shaped movable structures), from which the one or more robotic arms 304 may emanate (extend). The carriage(s) 312 may translate along a vertical column interface that runs at least a portion of the length of the column 310 to provide different vantage points from which the robotic arms 304 may be positioned to reach the patient 104. The carriage(s) 312 may rotate around the column 310 in some embodiments using a mechanical motor positioned within the column 310 to allow the robotic arms 304 to have access to multiples sides of the table 302. Rotation and/or translation of the carriage(s) 312 can allow the system 300 to align the medical instruments, such as endoscopes and/or catheters, into different access points on the patient 104. By providing vertical adjustment, the robotic arms 304 can be configured to be stowed compactly beneath the platform of the table system 300 and subsequently raised during a procedure. The robotic arms 304 may be mounted on the carriage(s) 312 through one or more arm mounts 314, which may comprise a series of joints that may individually rotate and/or telescopically extend to provide additional configurability to the robotic arms 304. The column 310 structurally provides support for the table platform and a path for vertical translation of the carriage(s) 312. The column 310 may also convey power and control signals to the carriage(s) 312 and/or the robotic arms 304 mounted thereon.

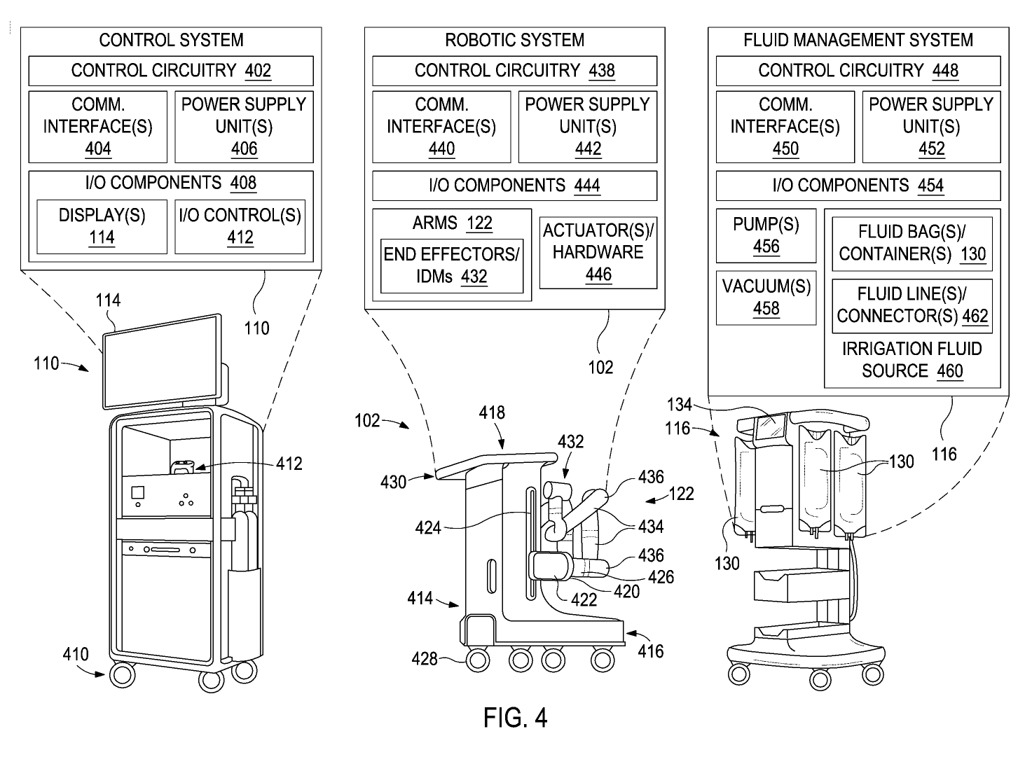

FIG. 4 illustrates medical system components that may be implemented in any of the medical systems of FIGS. 1-3 in accordance with one or more embodiments of the present disclosure. Although certain components are shown in FIG. 4, it should be understood that additional components not shown can be included in embodiments in accordance with the present disclosure. Furthermore, any of the illustrated components can be omitted, interchanged, and/or integrated into other devices/systems, such as the table 120, a medical instrument, etc.

The control system 110 can include one or more of the following components, devices, modules, and/or units (referred to herein as “components”), either separately/individually and/or in combination/collectively: control circuitry 402, one or more communication interfaces 404, one or more power supply units 406, one or more I/O components 408, and/or one or more mobilization components 410 (e.g., casters or other types of wheels). In some embodiments, the control system 110 can comprise a housing/enclosure configured and/or dimensioned to house or contain at least part of one or more of the components of the control system 110. In this example, the control system 110 is illustrated as a cart-based system that is movable using the mobilization components 410. In some cases, after reaching the appropriate position, the mobilization components 410 can be immobilized using wheel locks to hold the control system 110 in place. However, the control system 110 can be implemented as a stationary system, integrated into another system/device, and so on.

The one or more power supply units 406 can be configured to manage and/or provide power for the control system 110 (and/or the robotic system 102/fluid management system 116, in some cases). In some embodiments, the power supply units 406 include one or more batteries, such as a lithium-based battery, a lead-acid battery, an alkaline battery, and/or another type of battery. That is, the power supply units 406 can comprise one or more devices and/or circuitry configured to provide a source of power and/or provide power management functionality. Moreover, in some embodiments the power supply units 406 include a mains power connector that is configured to couple to an alternating current (AC) or direct current (DC) mains power source.

The one or more I/O components/devices 408 can include a variety of components to receive input and/or provide output, such as to interface with a user to assist in performing a medical procedure. The I/O components 408 can be configured to receive touch, speech, gesture, or any other type of input. In examples, the I/O components 408 can be used to provide input regarding control of a device/system, such as to control the robotic system 102, navigate a scope/catheter or other medical instrument attached to the robotic system 102 and/or deployed through the scope, control the table 120, control a fluoroscopy device, and so on. For example, a physician (not illustrated) can provide input via the I/O component(s) 408 and, in response, the control system 110 can send control signals to the robotic system 102 to manipulate a medical instrument. In examples, the physician can use the same I/O device to control multiple medical instruments (e.g., switch control between the instruments).

As shown, the I/O components 408 can include the one or more displays 114 (sometimes referred to as “the one or more display devices 114”) configured to display data. In some embodiments, the displays 114 include one or more touchscreens configured to receive input and/or display data. Further, the I/O components 408 can include one or more I/O devices/controls 412, which can include a touch pad, controller (e.g., hand-held controller, video-game-type controller, finger-based controls that enable finger-like movement, etc.), mouse, keyboard, wearable device (e.g., optical head-mounted display), virtual or augmented reality device (e.g., head-mounted display), foot panel (e.g., buttons at the user's feet), etc.

In some embodiments, the control system 110 can be coupled to the robotic system 102, a table 120, 302 (FIGS. 1-3) or another table, and/or a medical instrument, through one or more cables or connections (not shown). In some implementations, support functionality from the control system 110 can be provided through a single cable, simplifying and de-cluttering an operating room. In other implementations, specific functionality can be coupled in separate cabling and connections.

The robotic system 102 generally includes an elongate support structure 414 (also referred to as a “column”), a robotic system base 416, and a console 418 at the top of the column 414. The column 414 can include one or more carriages 420 (also referred to as “arm supports 420”) for supporting the deployment of one or more of the robotic arms 122. The carriage 420 can include individually configurable arm mounts that rotate along a perpendicular axis to adjust the base of the robotic arms 122 for positioning relative to a patient. The carriage 420 also includes a carriage interface 422 that allows the carriage 420 to vertically translate along the column 414. The carriage interface 422 can be connected to the column 414 through slots, such as slot 424, that are positioned on opposite sides of the column 414 to guide the vertical translation of the carriage 420. The slot 424 can include a vertical translation interface to position and/or hold the carriage 420 at various vertical heights relative to the base 416. Vertical translation of the carriage 420 allows the robotic system 102 to adjust the reach of the robotic arms 122 to meet a variety of table heights, patient sizes, physician preferences, etc. Similarly, the individually configurable arm mounts on the carriage 420 allow a robotic arm base 426 of the robotic arms 122 to be angled in a variety of configurations. The column 414 can internally comprise mechanisms, such as gears and/or motors, that are designed to use a vertically aligned lead screw to translate the carriage 420 in a mechanized fashion in response to control signals generated in response to user inputs, such as inputs from an I/O device(s).

The base 416 can balance the weight of the column 414, the carriage 420, and/or robotic arms 122 over a surface, such as the floor. Accordingly, the base 416 can house heavier components, such as one or more electronics, motors, power supply, etc., as well as components that enable movement and/or immobilize the robotic system 102. For example, the base 416 can include rollable wheels 428 (also referred to as “casters 428” or “mobilization components 428”) that allow for the robotic system 102 to move around the room for a procedure. After reaching an appropriate position, the casters 428 can be immobilized using wheel locks to hold the robotic system 102 in place during the procedure. As shown, the robotic system 102 also includes a handle 430 to assist with maneuvering and/or stabilizing the robotic system 102. In this example, the robotic system 102 is illustrated as a cart-based system that is movable. However, the robotic system 102 can be implemented as a stationary system, integrated into a table, and so on.

The robotic arms 122 can generally comprise the robotic arm bases 426 and end effectors 432, separated by a series of linkages 434 (also referred to as “arm segments 434”) that are connected by a series of joints 436. Each joint 436 can comprise an independent actuator and each actuator can comprise an independently controllable motor. Each independently controllable joint 436 represents an independent degree of freedom available to the robotic arm 122. In some embodiments, the end effectors 432 can be configured to engage with and/or control a medical instrument, a device, an object, and so on. The freedom of movement of the arms 122 can allow the robotic system 102 to position and/or direct a medical instrument from a desired point in space and/or allow a physician to move the arms 122 into a clinically advantageous position away from the patient to create access, while avoiding arm collisions.

The end effector 432 of each robotic arm 122 can comprise an instrument device manipulator (IDM). In some embodiments, the IDM can be removed and replaced with a different type of IDM. For example, a first type of IDM can manipulate an endoscope, a second type of IDM can manipulate a catheter, a third type of IDM can hold an EM field generator, and so on. However, the same IDM can be used to undertake some or all of the foregoing operations.

In some embodiments, the robotic arms 122 can be configured to control a position, orientation, and/or articulation of a medical instrument (e.g., a sheath and/or a leader of a scope) attached thereto. For example, the robotic arms 122 can be configured/configurable to manipulate a scope/catheter using elongate movement members. The elongate movement members can include one or more pull wires, cables, fibers, and/or flexible shafts. To illustrate, the robotic arms 122 can be configured to actuate multiple pull wires of the scope/catheter to deflect the tip of the scope/catheter. Pull wires can include any suitable or desirable materials, such as metallic and/or non-metallic materials such as stainless steel, Kevlar, tungsten, carbon fiber, and the like. In some embodiments, the scope/catheter is configured to exhibit nonlinear behavior in response to forces applied by the elongate movement members. The nonlinear behavior can be based on stiffness and/or compressibility of the scope/catheter, as well as variability in slack or stiffness between different elongate movement members.

The console 418 can include a display(s) to provide a user interface for receiving user input and/or providing output (e.g., a dual-purpose device, such as a touchscreen), such as to provide a physician/user with pre-operative data, intra-operative data, information to configure the robotic system 102, and so on. The console 418 can be positioned and tilted to allow a physician to access the console 418 from the side of the column 414 opposite arm base 426. From this position, the physician may view the console 418, robotic arms 122, and patient while operating the console 418 from behind the robotic system 102.

The robotic system 102 can also include control circuitry 438, one or more communication interfaces 440, one or more power supply units 442, one or more input/output components 444, and one or more actuators/hardware 446. The communication interfaces 440 can be configured to communicate with one or more device/sensors/systems. For example, the communication interfaces 440 can send/receive data in a wireless and/or wired manner over a network. The one or more power supply units 442 can be configured to manage and/or provide power for the robotic system 102. In some embodiments, the supply units 442 include one or more batteries, such as a lithium-based battery, a lead-acid battery, an alkaline battery, and/or another type of battery.

The one or more I/O components/devices 444 can be configured to receive input and/or provide output, such as to interface with a user. The I/O components 444 can be configured to receive touch, speech, gesture, or any other type of input. In examples, the I/O components 444 can be used to provide input regarding control of a device/system, such as to control/configure the robotic system 102. The I/O components 444 can include the one or more displays configured to display data.

Further, the I/O components 444 can include a touch pad, controller, mouse, keyboard, wearable device (e.g., optical head-mounted display), virtual or augmented reality device (e.g., head-mounted display), etc. Additionally, the I/O components 444 can include one or more speakers configured to output sounds based on audio signals and/or one or more microphones configured to receive sounds and generate audio signals. In some embodiments, the I/O components 444 include or are implemented as the console 418. Further, the I/O components 444 can include one or more buttons that can be physically pressed, such as a button on a distal end of a robotic arm 122 (which can enable/disable an admittance control mode of the robotic arm 122 for manual manipulation/movement of the robotic arm 122).

The one or more actuators/hardware 446 can be configured to facilitate movement of the robotic arms 122. Each actuator 446 can comprise a motor, which can be implemented in a joint or elsewhere within a robotic arm 122 to facilitate movement of the joint and/or a connected arm segment/linkage.

The robotic fluid management system 116 can include control circuitry 448, one or more communication interfaces 450, one or more power supply units 452, one or more input/output components 454, one or more pumps 456, one or more vacuums 458, and an irrigation fluid source 460. The communication interfaces 450 can be configured to communicate with one or more device/sensors/systems. For example, the communication interfaces 450 can send/receive data in a wireless and/or wired manner over a network.

The one or more power supply units 452 can be configured to manage and/or provide power for the fluid management system 116. In some embodiments, the power supply units 452 include one or more batteries, such as a lithium-based battery, a lead-acid battery, an alkaline battery, and/or another type of battery. That is, the power supply units 452 can comprise one or more devices and/or circuitry configured to provide a source of power and/or provide power management functionality.

The one or more I/O components/devices 454 can be configured to receive input and/or provide output, such as to interface with a user. The I/O components 454 can be configured to receive touch, speech, gesture, or any other type of input. The I/O components 454 can include a display, a touch pad, controller, mouse, keyboard, wearable device (e.g., optical head-mounted display), virtual or augmented reality device (e.g., head-mounted display), speaker, microphone, etc. Further, the I/O components 454 can include one or more buttons that can be physically pressed.

The fluid management system 116 can be configured to control the pump(s) 456 and/or the vacuum(s) 458 to provide irrigation/aspiration. For example, a medical instrument may be attached to the pump(s) 456/vacuum 458 to provide irrigation/aspiration to a target site via medical instrument.

The pump(s) 456 can be attached to an irrigation fluid source 460, which can include the fluid bag(s)/container(s) 130 and/or a fluid line(s)/connector(s) 462 to connect to a medical instrument(s). The pump(s) 456 can pump irrigation fluid (e.g., saline solution) through one or more medical instruments and into a treatment site. In some embodiments, the pump(s) 456 can be replaced with a vacuum that is configured to apply a vacuum pressure to draw the irrigation fluid from the irrigation fluid source 460 and out through the respective coupled medical instrument.

The vacuum(s) 458 can be configured to facilitate fluid aspiration. For example, the vacuum(s) 458 can be configured to apply a negative pressure to draw fluid out of a treatment site. The vacuum(s) 458 may be connected to a collection container into which withdrawn fluid is collected.

Example input data that can be used to generate position/orientation data for an object/medical instrument can include: sensor data from a sensor associated with a medical instrument (e.g., EM field sensor data, vision/image data captured by an imaging device/depth sensor/radar device on the medical instrument, accelerometer data from an accelerometer on the medical instrument, gyroscope data from a gyroscope on the medical instrument, satellite-based positioning data from a satellite-based sensor (a global positioning system (GPS), for example), and so on); feedback data from a robotic arm/component (also referred to as “kinematics data”) (e.g., data indicating how a robotic arm/component moved/actuated); robotic command data for a robotic arm/component (e.g., a control signal sent to the robotic system 102/robotic arm 122 to control movement of the robotic arm 122/medical instrument); shape sensing data from a shape sensing fiber (which can provide information regarding a location/shape of a medical instrument); model data regarding anatomy of a patient (e.g., a model of an interior/exterior portion of anatomy of the patient); position data of a patient (e.g., data indicating how the patient is positioned on a table); pre-operative data; etc.

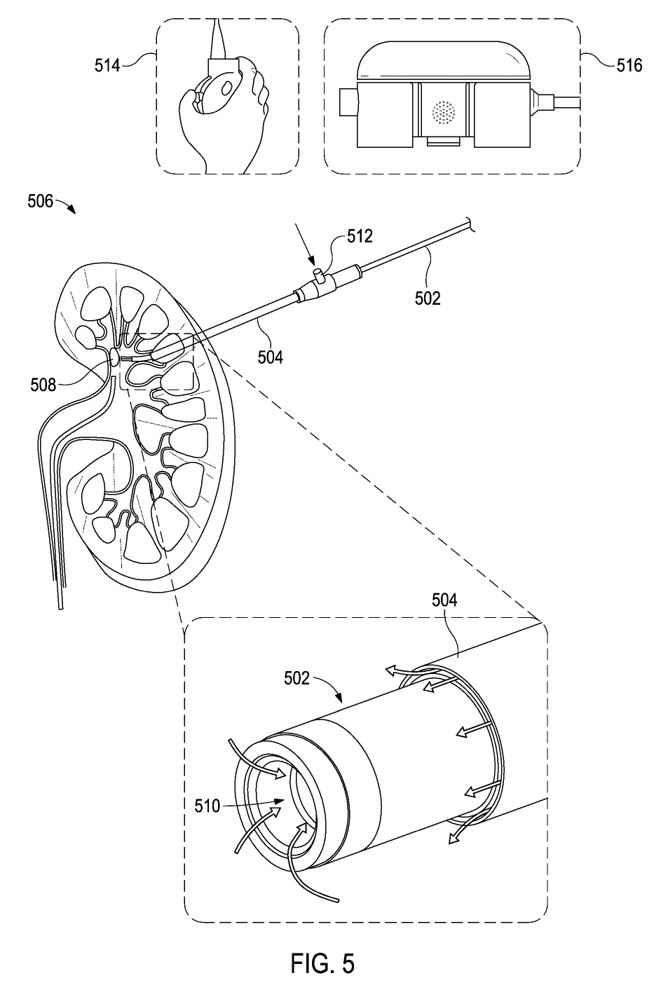

FIG. 5 illustrates an example catheter 502 and a percutaneous-access device 504 disposed at least partly in a kidney 506 of a patient in accordance with one or more embodiments. The catheter 502 and percutaneous-access device 504 may be representative of any of the catheters and percutaneous-access devices discussed herein. In this example, the catheter and percutaneous-access device 502, 504 are illustrated in the context of a urology procedure to treat/remove a kidney stone 508 from the kidney 506. However, the catheter and percutaneous-access device or “instruments” 502, 504 can be used in other types of procedures. As noted above, urology procedures and/or other types of procedures can be implemented manually at least in part and/or can be performed using robotic technologies at least in part.

The catheter 502 can be configured to be articulated, such as with respect to at least a distal end/tip of the catheter 502. For instance, the distal end portion/tip of the catheter 502 can be deflected in a variety of directions. In examples, the catheter 502 can be configured to move with two degrees of freedom (2-DOF) (e.g., two of x, y, z, yaw, pitch, or roll movement). To illustrate, the distal end portion of the catheter 502 can be configured to move right/left or up/down (e.g., x, y, or z movement) and also move to insert/retract the catheter 502 (e.g., translate along the x, y, or z axis). In other examples, the catheter 502 can be configured to move with 3-DOF (e.g., three of x, y, z, yaw, pitch, or roll movement). To illustrate, the distal end portion of the catheter 502 can be configured to move right/left and up/down (e.g., two of x, y, or z movement) and also move to insert/retract the catheter 502. However, the catheter 502 can also be configured to move with 4-DOF (e.g., x, y, z, and pitch/yaw/roll movement), 6-DOF (e.g., x, y, z, pitch, yaw, and roll movement), and so on. In some embodiments, such as when the catheter 502 is implemented with a robotically controllable handle, the catheter 502 is not configured for roll movements. However, the catheter 502 can be configured for roll and/or other types of movement in some cases, such as when the catheter 502 is configured with a manually controllable handle or some robotically controllable cases.

As shown, the catheter 502 can be implemented with the percutaneous-access device 504 to provide aspiration/irrigation to the kidney 506. The percutaneous-access device 504 may include one or more sheaths and/or shafts through which instruments (e.g., the catheter 502) and/or fluids may access the target anatomy in which the distal end of the device 504 is disposed. In some embodiments, active aspiration/suction may be drawn through a lumen 510 of the catheter 502 to a proximal end of the catheter 502 (e.g., a handle of the catheter 502). Further, in some embodiments, irrigation can be provided via the percutaneous-access device 504, such as between concentric sheaths. For example, a fluid management system (not illustrated) can be connected to an irrigation port 512 to provide irrigation to the percutaneous-access device 504, which travels down the percutaneous-access device 504 to the target site. FIG. 5 illustrates an example of the flow of aspiration fluid into the lumen 510 of the catheter 502 and the flow of irrigation fluid from the percutaneous-access device 504. In some embodiments, a passive aspiration outflow channel may be formed in the space between the outer wall of the catheter 502 and an inner wall/sheath of the percutaneous-access device/assembly 504. When the catheter 502 is disposed within the percutaneous-access device 504, the catheter 502 and the shaft(s)/sheath(s) of the percutaneous-access device 504 may be generally concentric. The catheter 502 and the percutaneous access device 504 may have generally circular cross-sectional shapes over at least portions thereof.

The catheter 502 may be controllable in any suitable or desirable way, either based on manual control and/or robotic control. In FIG. 5, handles/ bases 514, 516 provide examples that may be used to control the catheter 502. The first handle 514 illustrates a hand-held/manual handle that is configured to be manipulated by a physician/user to control movement of the catheter 502. Meanwhile, the second handle 516 illustrates a robotically controllable handle that is configured to be manipulated by a robotic arm, such as an end effector of a robotic arm, to control movement of the catheter 502. Example robotically controllable and manually controllable catheters are discussed in further detail below. By implementing an articulable catheter, the techniques/structures can allow various positions within the patient to be reached in a manner that prevents/minimizes damage to the anatomy of the patient. For example, a physician can navigate the distal portion of the catheter 502 to reach a particular cavity in the kidney 506 (e.g., calyx) where a kidney stone is located, without repositioning the rest of the shaft of the catheter 502 and/or the percutaneous-access device 504.

In embodiments, the catheter 502 is free of an imaging device. That is, the catheter 502 is implemented without an imaging device/camera on a distal end to capture image data of an internal anatomy of the patient. However, in other embodiments the catheter 502 can include an imaging device(s), such as on the tip of the catheter 502. Further, in embodiments, the catheter 502 is implemented without a position sensor (i.e., does not include a position sensor). However, the catheter 502 can be implemented with a position sensor in some cases, such as on a distal end of the catheter 502.

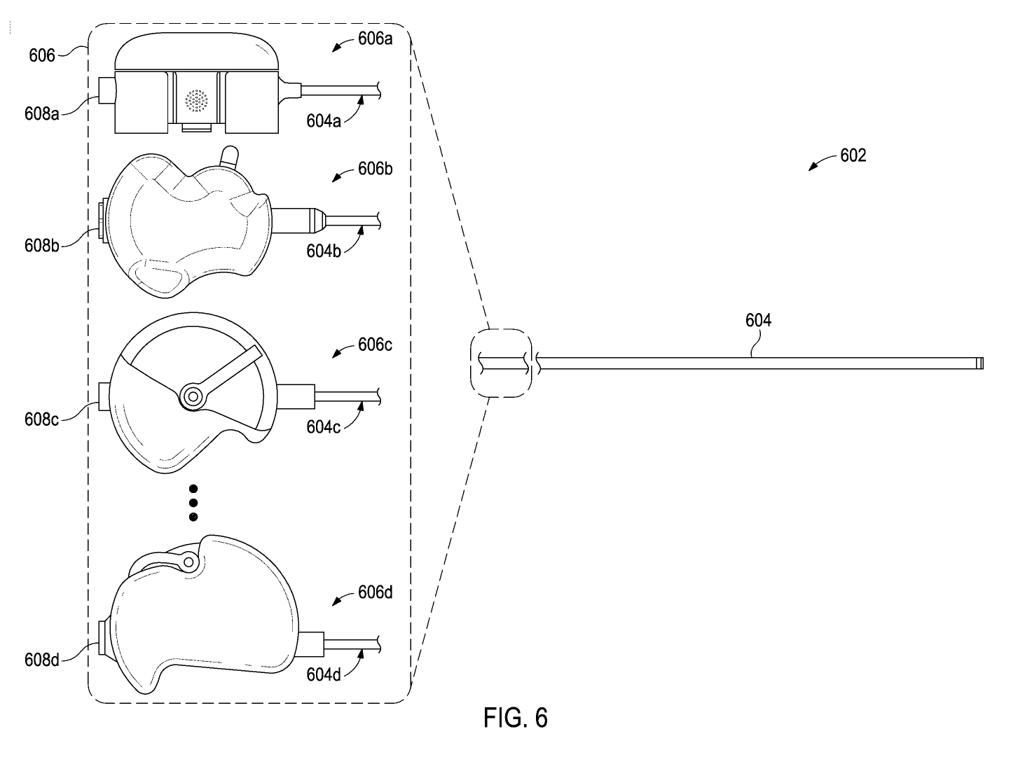

FIGS. 6, 7A-7B, and 8 illustrate example features of a robotically / manually controllable catheter 602 in accordance with one or more embodiments of the present disclosure. The features of the catheter 602 may be implemented in the context of one or more of the catheters discussed herein. As illustrated, the catheter 602 includes an elongate shaft 604 connected to a handle/base 606 (also referred to as the “instrument base 606”) that is configured to control actuation of at least a portion of the elongate shaft 604.

As shown in FIG. 6, the handle 606 can be implemented as a robotically controllable handle (e.g., the handle 606a) configured to couple to a robotic arm, and/or a manually controllable handle (e.g., the handles 606b, 606c, and 606d) configured to be held/manipulated by a user. In some embodiments, the elongate shaft 604 can extend through the handle 606 to a port 608 of the handle 606, which can be connected to a fluid management system and/or another system to facilitate aspiration, irrigation, deployment of an instrument through a working channel of the catheter 602, and so on.

Although certain handles are discussed in the context of being implemented in a manually-controllable catheter or robotically-controllable catheter, such catheters can be implemented in other contexts. For example, a manually-controllable catheter can include robotic components to be implemented as a robotically-controllable catheter (e.g., secondary use as a robotic catheter), and/or a robotically-controllable catheter can include manual component to be implemented as a manually-controllable catheter (e.g., secondary use as a manual catheter). As such, in some cases, a catheter is configured for both manual and robotic manipulation.

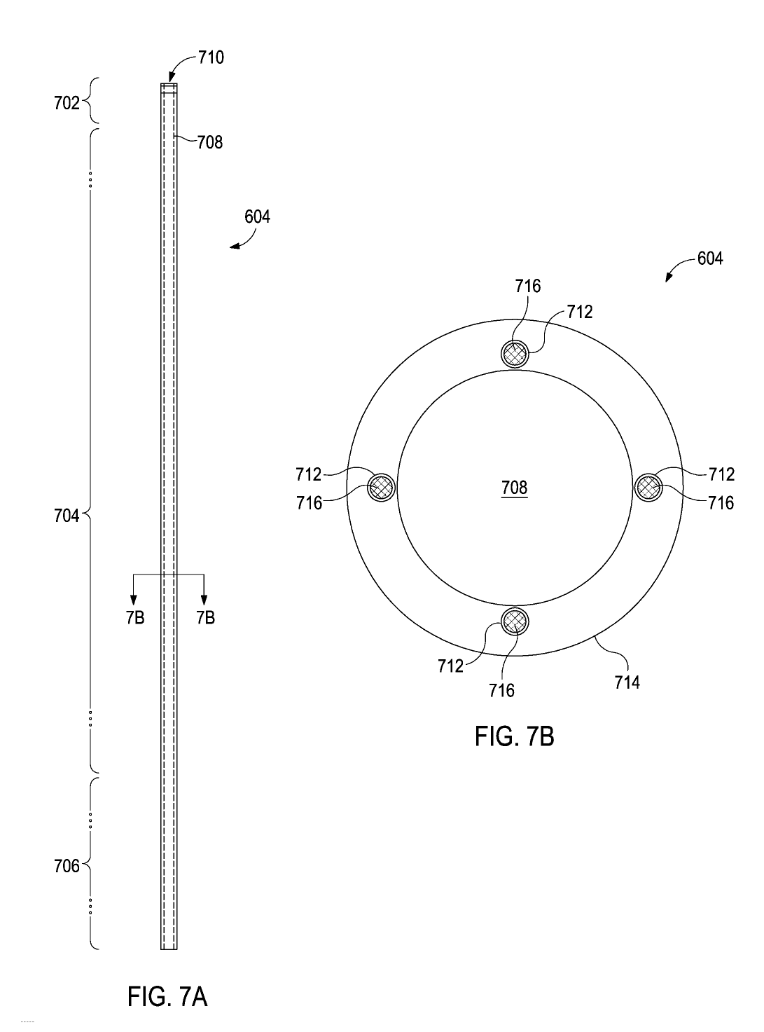

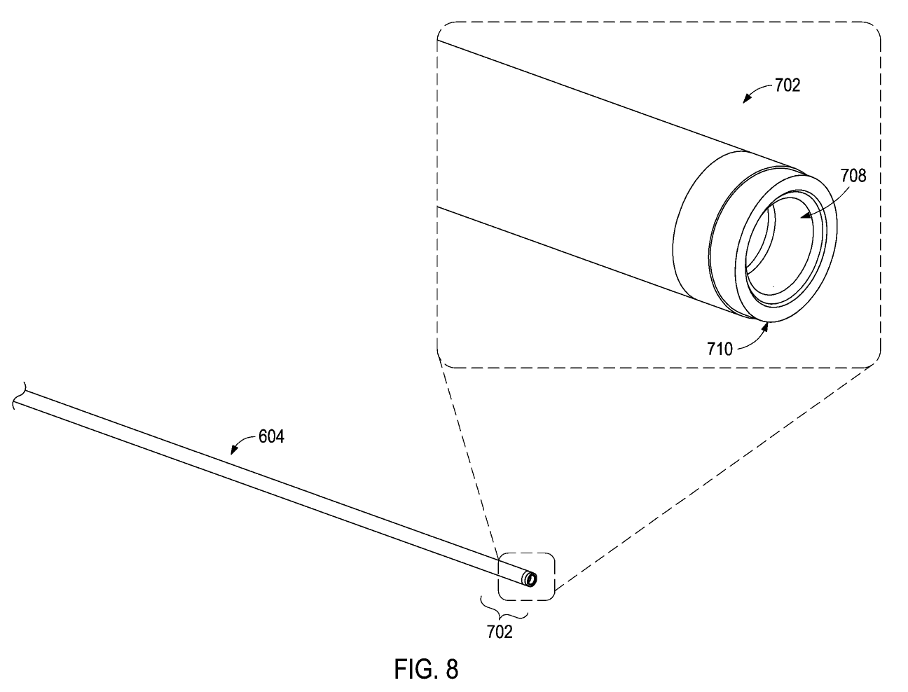

As shown in FIGS. 7A-7B and 8, the shaft 604 can include a distal/tip section/portion 702 (sometimes referred to as “the distal end portion 702”), a middle/medial section/portion 704, a proximal section/portion 706 (sometimes referred to as “the proximal end portion 706”), and a lumen 708 that extends through at least a portion of the shaft 604. For example, the lumen 708 can extend through the entirety of the shaft 604 from the distal section 702 (that may be positioned at a target site in a patient) to the proximal section 706 (that may be connected to the port 608 of the handle 606). However, the lumen 708 can extend another distance through the catheter 602. In examples, the lumen 708 can be referred to as a working channel. The distal section 702, the middle section 704, and/or the proximal section 706 can each be implemented with any longitudinal length. The terms distal, middle/medial, proximal, and/or other terms are used to describe a position of a feature relative to another feature. For example, a proximal feature of the catheter 602 can refer to a feature that is farthest from a target or anatomical site (e.g., during use/a procedure), whereas a distal feature of the catheter 602 can refer to a feature that is closest to the target or anatomical site.

As shown in FIG. 8, the distal section 702 of the shaft 604 can include a filter/containment structure/feature, referred to as a distal tip 710, that is configured to prevent certain objects from entering into the shaft 604 and/or configured to contain an object at a distal end of the shaft 604, such as when aspirating through the shaft 604. For example, in the context of a urological procedure, the distal section 702 of the catheter 602 can be positioned at a target site and used to aspirate one or more kidney stone fragments from a kidney. Here, the distal tip 710 can be configured to hold the kidney stone while the stone is being fragmented into pieces, such as by an instrument deployed from another device at the target site. The distal tip 710 can also prevent fragments that are larger than a particular size from being sucked into the rest of the shaft 604, which could clog the shaft 604 and impede/stop aspiration flow. Although the distal tip 710 is illustrated as a separate component from the rest of the shaft 604 (e.g., removably coupled to the rest of the shaft 604), in many examples the distal tip 710 can be integral with rest of the shaft 604 or implemented in other manners. Example features of the distal tip 710 are discussed in further detail below.

Portions of the shaft 604 can be formed of various materials, such as plastics, rubbers, vertebrae links, metal or plastic braids/coils, and so on, such that at least a portion of the shaft 604 is flexible for articulation. In some embodiments, the shaft 604 includes reinforcement material (e.g., braided) to strengthen and/or facilitate flexibility of the shaft 604. For example, the shaft 604 can include braid reinforcement for hoop strength and or to prevent kinking of the shaft 604 when the shaft 604 is navigated within the anatomy of a patient. Further, in some embodiments, the shaft 604 includes multiple layers of material that are implemented in a variety of configurations to facilitate the features of the shaft 604 discussed herein. In additional embodiments, the shaft 604 may be laser cut along its length to improve (increase) its flexibility.

In some cases, the distal tip 710 is formed of a different material than the rest of the shaft 604. For example, the distal tip 710 can be implemented with a material that avoids degradation in certain contexts, such as catastrophic degradation. The distal tip 710 can be implemented (e.g. made) with stainless steel (or other types of steel), titanium, tungsten, aluminum alloy, iron alloy, steel alloy, titanium alloy, tungsten alloy, and/or other materials (which may have relatively high melting points above a threshold) that can generally maintain its structure when laser beams inadvertently and/or occasionally contact the distal tip 710. However, the distal tip 710 and/or any other portion of the shaft 604 can be implemented with other materials. In some instances, the distal tip 710 includes certain materials that can have reduced degradation in certain contexts, such as when contacted by a laser. For example, the distal tip 710 can be formed of a material that has a fracture toughness greater than or equal to 2MPa·m1/2. However, other fracture toughness values/ranges can be implemented.

Referring again to FIGS. 7A-7B, and as shown in the cross-sectional view of FIG. 7B taken along the line shown in FIG. 7A, the shaft 604 can include one or more lumens 712 (also referred to as “wire lumens 712”) disposed in a wall 714 of the shaft 604, such as an outer wall. While four wire lumens 712 are shown, it will be appreciated that more or less than four wire lumens 712 may be included. In some embodiments, the lumens 712 are spaced equidistantly apart around the wall of the shaft 604, but could alternatively be non-equidistantly spaced. The catheter 602 can include one or more elongate movement members or “wires” 716 slidably disposed in the wire lumens 712 (also referred to as “wall lumens 712”). The wires 716 can include one or more pull wires, cables, fibers, and/or flexible shafts, and can be made of any suitable or desirable material such as, but not limited to, metallic and non-metallic materials, including stainless steel, Kevlar, tungsten, carbon fiber, and the like.

The catheter 602 is configured to exhibit nonlinear behavior in response to forces applied by the wires 716. The nonlinear behavior may be based on stiffness and/or compressibility of the catheter 602, as well as variability in slack or stiffness between different wires 716. Although a particular number of wire lumens 710 and wires 716 are illustrated in FIG. 7B, any number of lumens and/or wires can be implemented, without departing from the scope of the disclosure.

Steerable endoscopes, such as the catheter 602 of FIGS. 6-8, commonly use welding (e.g., laser welding, soldering, etc.) to connect the wires 716 to the distal end of the endoscope at the distal tip 710. In some cases, applying heat to the wires 716 in a welding or soldering process might degrade the mechanical properties of the wire, and may further reduce cross-sectional area of the wire as the wire material starts to reflow.

According to embodiments of the present disclosure, the wires 716 may be mechanically attached to the distal tip 710 in a manner that does not impact the material properties of the wire 716. As described in more detail below, the distal tip 710 may provide and otherwise define a wire groove or channel sized to receive the wire 716. Once the wire 716 is received within the wire channel, in some embodiments, a retaining structure (e.g., a ring, a flange, a sleeve, etc.) may be arranged over the interface between the wire and the groove and may be secured to the distal tip 710 using traditional or non-traditional methods at select points, thereby limiting (or entirely avoiding) the amount of heat introduced to the wires 716. The retaining structures may help hold the wires 716 in place within the grooves, thereby limiting movement of the wires 716 and preventing the wires 716 from dislodging from the distal tip 710.

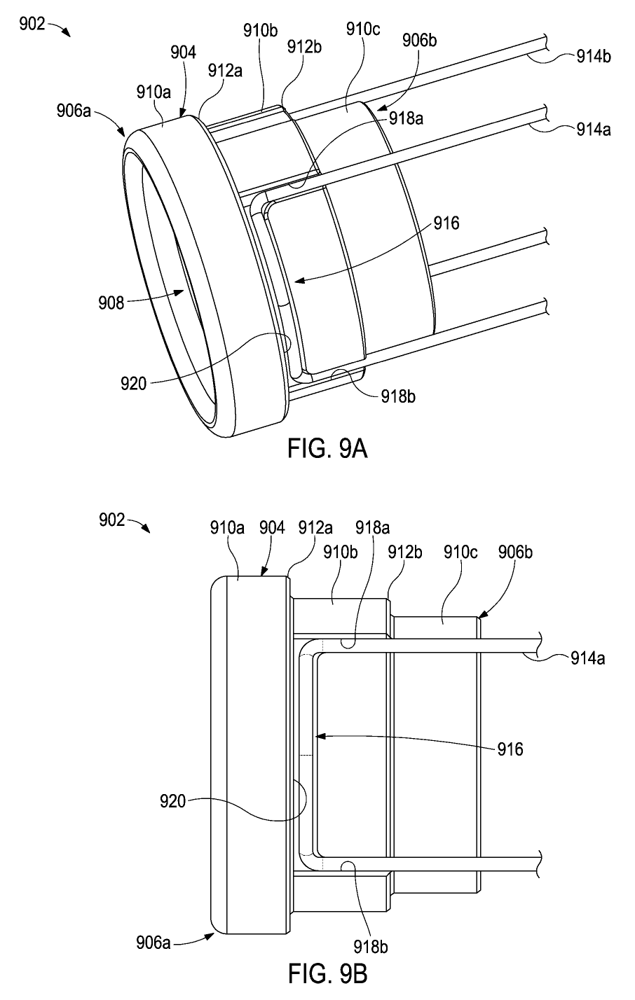

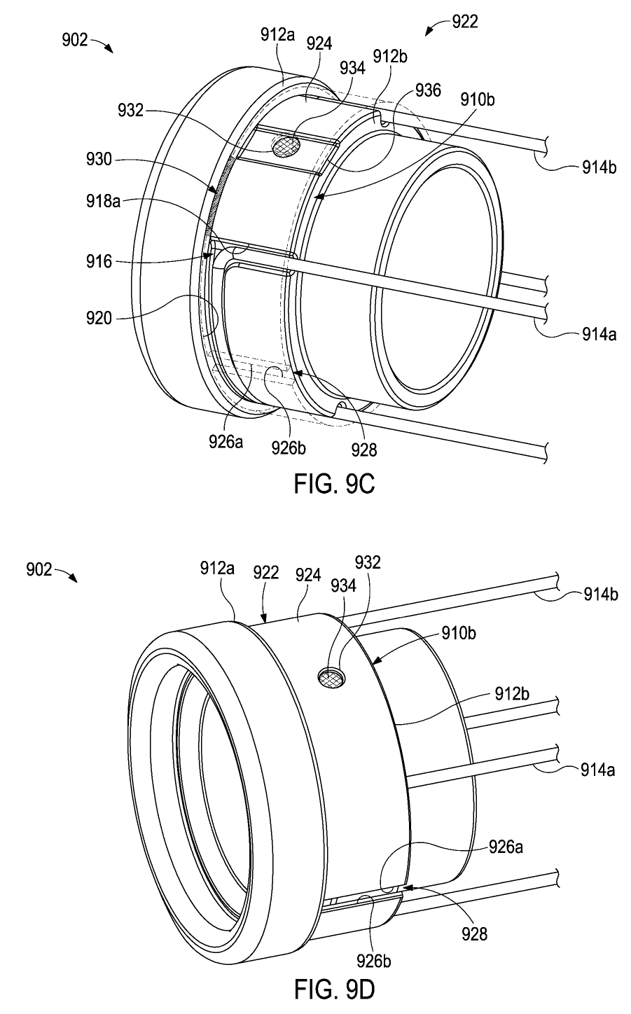

FIGS. 9A and 9B are enlarged isometric and side views, respectively, of an example distal tip 902 of a catheter, according to one or more embodiments. The distal tip 902 may be the same as or similar to the distal tip 710 used with the catheter 602 of FIGS. 6-8, and therefore, may be best understood with reference thereto. Accordingly, the distal tip 902 may be coupled to and otherwise arranged at the distal end of the shaft 604 (FIGS. 6 and 7A). It will be appreciated, however, that the distal tip 902 may be used with any of the other catheters or steerable shafts described herein, without departing from the scope of the disclosure.

As illustrated, the distal tip 902 provides a generally annular body 904 having a first or “distal” end 906a and a second or “proximal” end 906b opposite the distal end 906a. A central passageway 908 (FIG. 9A) is defined in the body 904 and extends between the distal and proximal ends 906a,b. The central passageway 908 fluidly communicates with the lumen 708 of FIGS. 7A-7B and 8 (e.g., the working channel) of the interconnected shaft 604 (FIGS. 6-8) and thereby helps facilitate aspiration/irrigation at a target site.

As illustrated, the body 904 provides and otherwise defines a first or “distal” portion 910a and a second or “intermediate” portion 910b contiguous with and extending proximally from the distal portion 910a. In at least one embodiment, the body 904 may further provide or define a third or “proximal” portion 910c contiguous with and extending proximally from the intermediate portion 910b. The intermediate portion 910b may exhibit a smaller diameter than the distal portion 910a, and the proximal portion 910c may exhibit a smaller diameter than the intermediate portion 910b. Accordingly, the body 904 may provide a first shoulder 912a at the interface between the distal and intermediate portions 910a,b, and a second shoulder 912b at the interface between the intermediate and proximal portions 910b,c.

As shown in FIGS. 9A and 9B, one or more wires 914a and 914b, alternatively referred to as “pull wires,” may be operatively coupled to the distal tip 902. The wires 914a,b may be the same as or similar to the wires 716 (FIG. 7B) extending within the wire lumens 712 (FIG. 7B) disposed in the wall 714 (FIG. 7B) of the shaft 604 (FIGS. 7A-7B) of the catheter 602 (FIGS. 6-8) and therefore may be best understood with reference thereto. The wires 914a,b may extend proximally from the distal tip 902 to one or more manipulation components in the handle 606 (FIG. 6) where the wires 914a,b can be manipulated to control movement of the distal end of the catheter 602 and thereby control articulation of the distal tip 902. More specifically, the handle 606 can be actuated (operated) to control movement of the distal tip 902, such as to articulate, advance, retract, or roll the distal tip 902.

The wires 914a,b can comprise wires, cables, fibers, and/or flexible shafts and can be made of any suitable or desirable material such as, but not limited to, metallic and non-metallic materials, including stainless steel, Kevlar, tungsten, carbon fiber, and the like. In the illustrated embodiment, two wires 914a,b are included, but any number of wires 914a,b can be implemented, without departing from the scope of the disclosure.

As illustrated, one or more wire grooves or channels 916 may be defined in the body 904 and sized to receive a corresponding one of the wires 914a,b. The first wire 914a is depicted as being received in a corresponding one of the wire channels 916, while the second wire 914b is received within a second wire channel 916, which is provided on the angular opposite side of the body 904 and occluded from view. As illustrated, the grooves 916 may be defined in the intermediate portion 910b.

In some embodiments, as illustrated, the wire channel 916 may define a generally C or U-shaped route or path that includes first and second axial legs 918a and 918b extending substantially parallel to the shaft 604 (FIGS. 7A-7B) or a longitudinal (central) axis of the body 904, and a transverse portion 920 that interconnects the axial legs 918a,b The transverse portion 920 may extend circumferentially about a segment of the intermediate portion 910b to interconnect the axial legs 918a,b. The axial legs 918a,b may generally terminate at the second shoulder 912b and axially align with the wire lumens 712 (FIG. 7B) of the shaft 604 through which the wires 914a,b extend. Consequently, the wires 914a,b can exit the wall 714 (FIG. 7B) of the shaft 604 and extend directly into the axial legs 918a,b without any bending or stressing of the wires 914a, b.

In the depicted embodiment, the first wire 914a is routed through the wire channel 916 by threading the first wire 914a through the first axial leg 918a, through the interconnected transverse portion 920, and through the second axial leg 918b such that opposing ends of the first wire 914a start and end at a proximal location, such as the handle 606 (FIG. 6). In other embodiments, however, the wire channel 916 may comprise other shapes such as, but not limited to, a substantially curved or arcuate route or path. By routing the wires 914a,b through the wire channels 916, the handle 606 can selectively apply tension to create reaction forces that facilitate antagonistic movement of the wires 914a,b and thereby enable articulation of the distal end of the catheter 602 (FIGS. 6-8).

While the wire channel 916 is described herein as exhibiting a generally C or U-shaped route or path, it is contemplated herein to include other path geometries including, but not limited to S-shaped curved, curvilinear path, or a tortuous path, without departing from the scope of the disclosure.

FIGS. 9C and 9D are isometric back and front views, respectively, of the distal tip 902 according to one or more additional embodiments. In some embodiments, as illustrated, the distal tip 902 may further include a capture ring 922 (shown in phantom in FIG. 9C) used to help secure the wires 914a,b to the distal tip 902 and otherwise maintain the wires 914a,b within the corresponding wire channels 916 (only visible in FIG. 9C). The capture ring 922 may provide a generally annular body 924 and may be sized to extend about the circumference of the intermediate portion 910b where the wire channels 916 are provided. In at least one embodiment, the body 924 may exhibit a width substantially similar to the width of the intermediate portion 910. In such embodiments, the body 924 may extend between the first and second shoulders 912a,b when mounted to the intermediate portion 910b. Accordingly, in at least one embodiment, the capture ring 922 may extend flush with the second shoulder 912b but could alternatively stop short of the second shoulder 912b in other embodiments.

In some embodiments, the body 924 may be continuous, annular structure that forms a full 360° turn. In other embodiments, however, the body 924 may be discontinuous and include opposing first and second ends 926a and 926b. When the capture ring 922 is mounted to the distal tip 902 at the intermediate portion 910b, a gap 928 may form between the ends 926a,b to accommodate the diameter (size) of the intermediate portion 910b. Having a discontinuous body 924 with opposing ends 926a,b may advantageously allow the capture ring 922 to be used with distal tips 902 having varying diameters. More specifically, the size of the gap 920 may increase when the capture ring 922 is mounted to an intermediate portion 910b having a larger diameter and may alternatively decrease when the capture ring 922 is mounted to an intermediate portion 910b with a smaller diameter.

Once the body 924 is properly seated on the distal tip 902 (e.g., at the intermediate portion 910b and covering the wires 914a,b received within the corresponding channels 916), the capture ring 922 then may be secured to the distal tip 902. In some embodiments, the capture ring 922 may be crimped onto the distal tip 902 at the intermediate portion 910b. In other embodiments, the capture ring 922 may be welded (e.g., laser welded) or soldered to the distal tip 902. In yet other embodiments, the capture ring 922 may be attached to the distal tip 902 using an adhesive. In even further embodiments, the capture ring 922 may be press fit onto the distal tip 902 or attached to the distal tip 902 using any combination of the foregoing.

In embodiments where the capture ring 922 is welded or soldered to the distal tip 902, one or more butt welds may be applied at the interface between the body 924 and the first shoulder 912a, thereby resulting in a corresponding one or more weld interfaces 930 (one shown in FIG. 9A). In some embodiments, the capture ring 922 may include a plurality of welded interfaces 930 at the interface between the body 924 and the first shoulder 912a and may be spaced (equidistantly or non-equidistantly) about the circumference of the capture ring 922. In other embodiments, however, the welded interface 930 may extend about the entire circumference of the capture ring 922 at the interface between the body 924 and the first shoulder 912a. Alternatively, or in addition thereto, the capture ring 922 may be welded to the distal tip 902 at the interface between the body 924 and the second shoulder 912a, without departing from the scope of the disclosure.

In some embodiments, as illustrated, the capture ring 922 may provide or otherwise define one or more weld apertures 932 where the capture ring 922 may be welded or soldered to the distal tip 902, thereby resulting in a welded interface 934. In embodiments with multiple weld apertures 932, the weld apertures 932 may be equidistantly or non-equidistantly spaced from each other about the circumference of the capture ring 922. Moreover, in at least one embodiment, the weld apertures 932 may be aligned centrally along the width of the capture ring 922. More specifically, when the capture ring 922 is arranged about the distal tip 902 (e.g., at the intermediate portion 912b), the weld apertures 932 may be positioned at or near a midway point between the first and second shoulders 912a,b. In other embodiments, however, one or more of the weld apertures 932 may alternatively be positioned closer to either of the shoulders 912a,b, without departing from the scope of the disclosure.

The capture ring 922 may be arranged about the distal tip 902 such that the weld apertures 932 are positioned away from and do not overlap (are angularly offset from) the wire channels 916. In particular, the capture ring 922 may be arranged such that the weld apertures 932 are not positioned radially outward from the wire channels 916 but are instead angularly spaced (offset) from the wire channels 916. As will be appreciated, this may be advantageous in preventing heat stress generated during a welding (or soldering) process from adversely affecting the wires 914a,b, and thereby weakening the mechanical properties of the wires 914a,b. More specifically, the mechanical properties of the wires 914a,b may be degraded if the wires 914a,b are exposed to heat associated with welding or soldering, or the cross-sectional area of the wires 914a,b may be reduced if the material begins to flow during welding or soldering.

In some embodiments, the weld apertures 932 may angularly interpose the wire channels 916, such as being centrally located between the wire channels 916, or alternatively angularly closer to one or the other of the wire channels 916. In other embodiments, however, one or more of the weld apertures 932 may be nested (arranged) within a corresponding one of the wire channels 916 but not overlapping any portion of the wire channel 916. More specifically, in such embodiments, the weld apertures 932 may angularly interpose the first and second axial legs 918a,b (see also FIGS. 9A-9B) of a corresponding wire channel 916, but may also be offset from (not overlapping) the transverse portion 920. Accordingly, in such embodiments, the weld aperture 932 is spaced from each section of the wire channel 916; e.g., the axial legs 918a,b and the transverse portion 920.

The weld apertures 932 may allow the capture ring 922 to be welded to the intermediate portion 910b at targeted locations and facilitate optimal reflow and strength. In some embodiments, as best seen in FIG. 9C, at least one of the weld apertures 932 may be angularly aligned with a depression or pocket 936 defined in the distal tip 902 (e.g., in the intermediate portion 910b). As illustrated, the pocket 936 may comprise a recess that extends axially along at least a portion of the width of the intermediate portion 910b. As welding or soldering material is added at the corresponding weld apertures 932, the material may flow through the weld aperture 932 and into the pocket 936, thereby resulting in stronger interconnection between the capture ring 922 and the distal tip 902. In addition, the weld apertures 932 may be used to help reflow a polymer jacket, such as the polymer sheath described below, into the pocket 936, thereby allowing the sheath to be bonded to the capture ring 922.

Accordingly, securing (e.g., welding) the capture ring 922 to the intermediate portion 910 at the weld apertures 932 may prevent slippage between the capture ring 922 and the intermediate portion 910. In at least one embodiment, the capture ring 922 is made of stainless steel and is the same material as the intermediate portion 910. In other embodiments, the capture ring 922 may comprise a different material from the intermediate portion 910 and can include any suitable or desirable material, such as metallic and non-metallic materials, including stainless steel, Kevlar, tungsten, carbon fiber, plastics, nylons, polymers, etc., without departing from the scope of the disclosure.

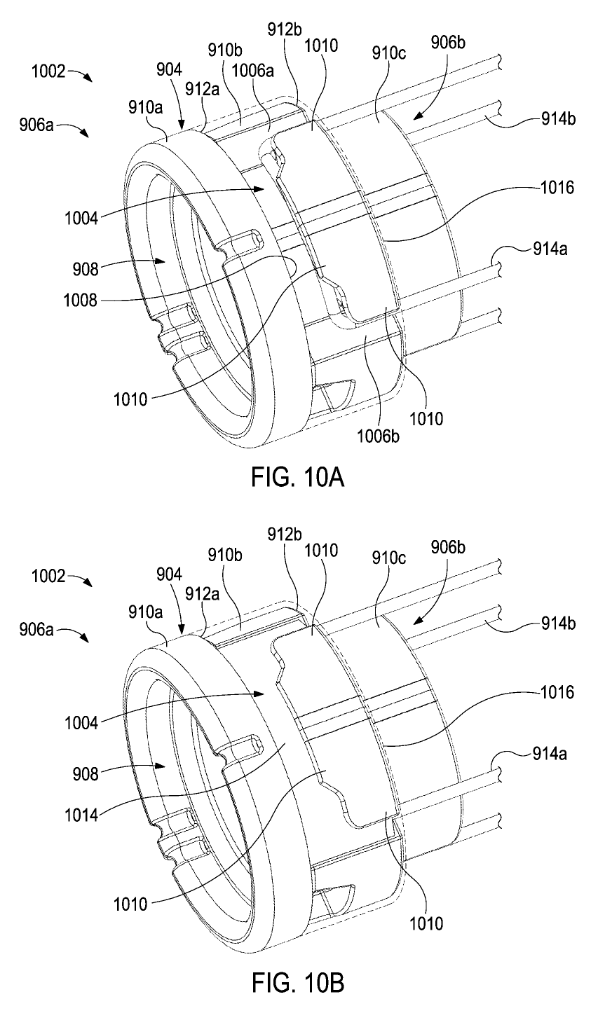

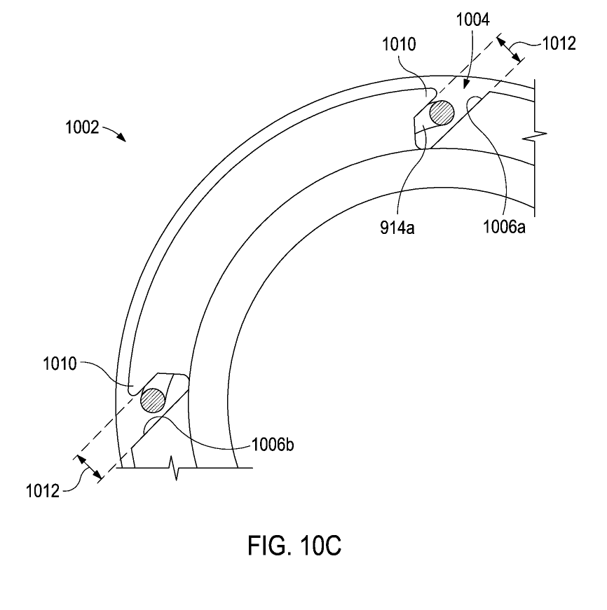

FIGS. 10A and 10B are enlarged isometric and side views, respectively, of another example distal tip 1002 of a catheter, according to one or more additional embodiments. The distal tip 1002 may be similar in some respects to the distal tip 902 of FIGS. 9A-9D, and therefore may be best understood with reference thereto.

Similar to the distal tip 902, for example, the distal tip 1002 may be coupled to and otherwise arranged at the distal end of the shaft 604 (FIGS. 6 and 7A). Moreover, the distal tip 1002 includes the annular body 904 with opposing distal and proximal ends 906a,b opposite the distal end 906a, and the central passageway 908 extending between the distal and proximal ends 906a,b. The body 904 also includes the distal, intermediate, and proximal portions 910a-c with differing diameters, thereby providing the first and second shoulders 912a,b, as generally described above.

The wires 914a,b may also be operatively coupled to the distal tip 1002. In particular, the wires 914a,b may be received within corresponding wire channels 1004 defined in the body 904 and sized to receive a corresponding one of the wires 914a,b. As illustrated, the wire channels 1004 may be defined in the intermediate portion 910b. The first wire 914a is depicted as being received in a corresponding one of the wire channels 1004, while the second wire 914b is received within a second wire channel 1004 provided on the angular opposite side of the body 904 and occluded from view.

In some embodiments, as illustrated, each wire channel 1004 may define a generally C or U-shaped route or path that includes first and second axial legs 1006a and 1006b extending substantially parallel to the shaft 604 (FIGS. 7A-7B) or a longitudinal (central) axis of the body 904, and a transverse portion 1008 that interconnects the axial legs 1006a,b and extends circumferentially about a segment (section) of the intermediate portion 910b. In the depicted embodiment, the first wire 914a is routed through the wire channel 1004 by threading the first wire 914a through the first axial leg 1006a, through the interconnected transverse portion 1008, and through the second axial leg 1006b such that opposing ends of the first wire 914a start and end at a proximal location, such as the handle 606 (FIG. 6). In other embodiments, however, the wire channel 1004 may comprise other shapes such as, but not limited to, a substantially curved or arcuate route or path.

Unlike the wire channels 916 of FIGS. 9A-9B, however, one or both of the wire channels 1004 may include one or more flanges or “overhang entrapment features” 1010 operable to help retain the wires 914a,b within the wire channels 916. The overhang entrapment features 1010 may comprise portions of the body 904 (e.g., the intermediate portion 910b) that extend into the wire channels 916, thereby forming a gap or open area radially between the overhang entrapment features 1010 and the bottom of the corresponding wire channel 1004. The gap formed by the overhang entrapment features 1010 may be large enough to receive and seat the wires 914a,b.

Referring briefly to FIG. 10C, illustrated as an axial end view of a portion of the distal tip 1002 showing at least one embodiment of the overhang entrapment features 1010. In particular, illustrated are overhang entrapment features 1010 that extend into the wire channel 1004 at the first and second axial legs 1006a,b. As illustrated, a gap 1012 is defined between the overhang entrapment features 1010 and the bottom of the corresponding wire channel 1004. Moreover, the first wire 914a may be received within the wire channel 104 and seated within the gap 1012 such that all or a portion of the overhang entrapment feature 1010 extends over the wire 914a and thereby helps retain the wire 914a within the wire channel 1004. The overhang entrapment features 1010 may be advantageous during the assembly process of the distal tip 1002 by helping to retain the wires 914a,b within the wire channel 1004 until the wires 914a,b are fully secured to the distal tip 1002. However, the overhang entrapment features 1010 may also prove advantageous during operation of the distal tip 1002.

Referring again to FIGS. 10A-10B, in some embodiments, once properly received within the corresponding wire channels 1004, the wires 914a,b be secured to the distal tip 1002 by crimping one or more of the overhang entrapment features 1010 onto the wires 914a,b. In other embodiments, or in addition thereto, the wires 914a,b may be attached to the distal tip 1002 via welding (e.g., laser welding) or soldering. In such embodiments, the welding process may not adversely affect the mechanical properties of the wires 914a,b because of the overhang entrapment features 1010. More specifically laser welding at the overhang entrapment features 1010 may allow the material of the overhang entrapment features 1010 to flow, but the material of the wires 914a,b will maintain relatively solid and instead be encapsulated by the molten weld material, thereby securing the wires 914a,b to the distal tip 1002 and preventing the wires 914a,b from being exposed to the outer diameter of the capture ring 922.

In yet other embodiments, or in addition to any of the foregoing methods, the wires 914a,b may be attached to the distal tip 1002 by applying or otherwise depositing a material 1014 within the weld channel 1004, as shown in FIG. 10B. In such embodiments, for example, the material 1012 may comprise an epoxy or another flowable material that, when hardens, not only encapsulates the wires 914a,b but also rigidly secures the wires 914a,b to the distal tip 1002, thereby preventing the wires 914a,b from being exposed to the outer diameter of the capture ring 922.

In some embodiments, a sleeve or sheath 1016 (shown in dashed lines) may be included in the distal tip 1002 and sized to fit over top and circumferentially cover the weld channel 1004 at the intermediate portion 910b. The sheath 1016 may be made of a variety of materials, such as plastic or metal, and may be secured to the distal tip 1002 through use of an adhesive, laser welding, laminating, soldering, etc. In at least one embodiment, the sheath 1016 may comprise a polymer material that is attached to the distal tip 1002 by heat shrinking or chemically bonded to the catheter. As will be appreciated, the sheath 1016 may help further secure the wires 914a,b from escaping from the corresponding weld channels 1004 and may additionally seal the distal end, thereby protecting the catheter from environmental hazards.

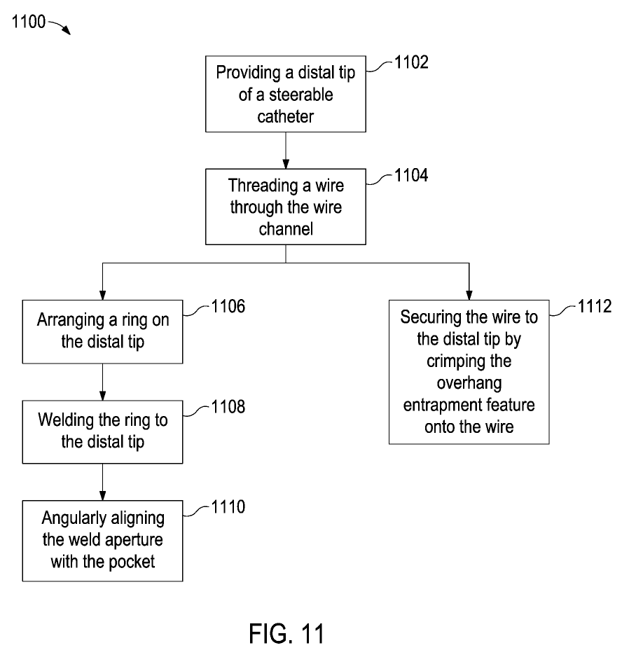

FIG. 11 is a schematic flowchart depicting an example method 1100 for assembling a distal tip of a steerable catheter, in accordance with the principles of the present disclosure. The method 1100 is applicable to the embodiments disclosed herein and, therefore, may be used to assemble the distal tip 902 according to FIGS. 9A-9D or the distal tip 1002 according to FIGS. 10A-10C. Accordingly, the method 1100 may be best understood with reference to the description of FIGS. 9A-9D and 10A-10C.

As illustrated, the method 1100 may include providing a distal tip of a steerable catheter, as at 1102. The distal tip comprises the annular body 904 having opposing first and second ends 906a,b, and the annular body 904 may have the distal portion 910a provided at or near the first end 906a and a second portion 910b contiguous with and extending proximally from the first portion 910a. The distal tip may further include a wire channel defined in the second portion, such as the wire channel 916 of FIGS. 9A-9D or the wire channel 1004 of FIGS. 10A-10C. The wire channel may define a path for the wires 914a,b and be sized to receive the wires 914a,b such that the opposing ends of the wires 914a,b extend proximally from the annular body. Moreover, the first portion 910a and the second portion 910b of the annular body 904 may each define diameters. In at least one embodiment, the diameter of the first portion 910a is larger than the diameter of the second portion 910b, such that a first shoulder, such as the first shoulder 912a of FIGS. 9A-9D, may be provided at an interface between the first and second portions 910a,b.

The method 1100 may further include loading or threading one or more wires 914a,b into the wire channel defined in the annual body 904, as at 1104. Because the wire channel, according to one or more embodiments, is defined on an outer facing surface of the annular body 904, the wires 914a,b need not be threaded through the wire channels. Rather, the wires 914a,b may be side loaded into the channel under tension, thereby easing the manufacturing and assembly process.

The method 1100 may then optionally further include mounting or arranging a ring (e.g., the capture ring 922 of FIGS. 9A-9D) to the annular body 904 at the second portion 910b, as at 1106. The ring may at least partially cover the wire channel. Moreover, in at least one embodiment, the ring may be discontinuous about the second portion 910b of the annular body 904 and thus may define a discontinuous annular body about the second portion 910b of the annular body 904 of the distal tip. In such embodiments, the ring may have opposing first and second ends.

After mounting the ring to the distal tip, as at 1106, the method 1100 may further include provide welding the ring to the distal tip, as at 1108. The ring may be welded at one or more weld interfaces. In some embodiments, the weld interfaces may comprise butt welds. The weld material may be applied at an interface between the ring and the first shoulder, according to embodiments. In at least one embodiment, the ring may comprise one or more weld apertures, such as the weld apertures 932 of FIGS. 9C and 9D. In such embodiments, welding the ring to the annular body 904 comprises welding the ring at the one or more weld apertures, thus the one or more weld apertures may define weld interfaces. In embodiments where the ring defines weld apertures, the ring may be arranged on the distal tip such that the one or more weld apertures are angularly offset from the wire channel.

As described herein, in some embodiments, the distal tip defines a pocket, such as the pocket 936 as shown in FIG. 9C. In such embodiments, the method 1100 further include angularly aligning at least one of the weld apertures with the pocket, as at 1110.

In an alternative embodiment, the annular body 904 may define an overhang entrapment feature, such as the overhang entrapment features 1010 of FIGS. 10A and 10B. The overhang entrapment feature may extend into the wire channel and thereby form a gap sized to receive the wire 914a,b. In such embodiments, the method 1100 may optionally include securing the wire 914a,b to the distal tip by crimping the overhang entrapment feature onto the wire 914a,b, as at 1112.

Embodiments disclosed herein include:

A. A steerable catheter including an elongate shaft having opposing distal and proximal ends, a wire extending within a wall of the elongate shaft between the distal and proximal ends, and a distal tip arranged at the distal end and including an annular body, and a wire channel defined in the annular body and providing a path sized to receive the wire and including first and second axial legs and a transverse portion interconnecting the first and second axial legs, wherein the wire is routed through the wire channel such that opposing ends of the wire extend proximally from the distal tip.

B. A distal tip for a steerable catheter including an annular body providing opposing first and second ends, a first portion provided at the first end, and a second portion contiguous with and extending proximally from the first portion, and a wire channel defined in the second portion in the form of a path and sized to receive a wire such that opposing ends of the wire extend proximally from the annular body, wherein a diameter of the first portion is larger than a diameter of the second portion such that a first shoulder is provided at an interface between the first and second portions.

Each of embodiments A and B may have one or more of the following additional elements in any combination: Element 1: wherein the annular body provides opposing first and second ends, a first portion provided at the first end, and a second portion contiguous with and extending proximally from the first portion, the wire channel being defined in the second portion, wherein a diameter of the first portion is larger than a diameter of the second portion such that a first shoulder is provided at an interface between the first and second portions. Element 2: wherein the annular body further provides a third portion contiguous with and extending proximally from the second portion, and wherein the diameter of the second portion is larger than a diameter of the third portion such that a second shoulder is provided at an interface between the second and third portions. Element 3: further comprising a ring mounted to the annular body and at least partially covering the wire channel with the wire received within the wire channel. Element 4: wherein the ring is secured to the distal tip by at least one of crimping, welding, soldering, and an adhesive. Element 5: wherein the ring is welded to the distal tip at one or more weld interfaces comprising butt welds applied at an interface between the ring and the distal tip. Element 6: wherein one or more weld apertures are defined in the ring and the ring is welded to the distal tip at the one or more weld apertures, and wherein the ring is arranged on the distal tip such that the one or more weld apertures are angularly offset from the wire channel. Element 7: wherein the annular body defines an overhang entrapment feature extending into the wire channel and thereby forming a gap sized to receive the wire. Element 8: wherein the wire is secured to the distal tip by crimping the overhang entrapment feature onto the wire. Element 9: wherein the wire is secured to the distal tip by welding at the overhang entrapment feature. Element 10: further comprising a material deposited within the weld channel to encapsulate the wire. Element 11: further comprising a sheath secured to the distal tip and arranged over top and circumferentially covering the weld channel. Element 12: wherein the annular body further provides a third portion contiguous with and extending proximally from the second portion, and wherein the diameter of the second portion is larger than a diameter of the third portion such that a second shoulder is provided at an interface between the second and third portions. Element 13: further comprising a ring mountable to the annular body at the second portion and at least partially covering the wire channel. Element 14: wherein the ring comprises a discontinuous annular body having opposing first and second ends. Element 15: wherein the ring is welded to the distal tip at one or more weld interfaces comprising butt welds applied at an interface between the ring and the first shoulder. Element 16: wherein one or more weld apertures are defined in the ring and the ring is welded to the distal tip at the one or more weld apertures, and wherein the ring is arranged on the distal tip such that the one or more weld apertures are angularly offset from the wire channel. Element 17: wherein a pocket is defined in the distal tip and the ring is arranged on the distal tip such that a corresponding one of the one or more weld apertures angularly aligns with the pocket. Element 18: wherein the annular body defines an overhang entrapment feature extending into the wire channel and thereby forming a gap sized to receive the wire, and wherein the wire is secured to the distal tip by at least one of crimping the overhang entrapment feature onto the wire, and welding at the overhang entrapment feature.

By way of non-limiting example, exemplary combinations applicable to A and B include: Element 1 with Element 2; Element 3 with Element 4; Element 3 with Element 5; Element 3 with Element 6; Element 7 with Element 8; Element 7 with Element 9; Element 7 with Element 10; Element 7 with Element 11; Element 13 with Element 14; Element 13 with Element 15; Element 13 with Element 16; Element 16 with Element 17.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A steerable catheter, comprising:
an elongate shaft having opposing distal and proximal ends;
a wire extending within a wall of the elongate shaft between the distal and proximal ends; and
a distal tip arranged at the distal end and including:
an annular body; and
a wire channel defined in the annular body and providing a path sized to receive the wire and including first and second axial legs and a transverse portion interconnecting the first and second axial legs,
wherein the wire is routed through the wire channel such that opposing ends of the wire extend proximally from the distal tip.

2. The steerable catheter of claim 1, wherein the annular body provides:
opposing first and second ends;
a first portion provided at the first end; and
a second portion contiguous with and extending proximally from the first portion, the wire channel being defined in the second portion,
wherein a diameter of the first portion is larger than a diameter of the second portion such that a first shoulder is provided at an interface between the first and second portions.

3. The steerable catheter of claim 2, wherein the annular body further provides a third portion contiguous with and extending proximally from the second portion, and wherein the diameter of the second portion is larger than a diameter of the third portion such that a second shoulder is provided at an interface between the second and third portions.

4. The steerable catheter of claim 1, further comprising a ring mounted to the annular body and at least partially covering the wire channel with the wire received within the wire channel.

5. The steerable catheter of claim 4, wherein the ring is secured to the distal tip by at least one of crimping, welding, soldering, and an adhesive.

6. The steerable catheter of claim 4, wherein the ring is welded to the distal tip at one or more weld interfaces comprising butt welds applied at an interface between the ring and the distal tip.

7. The steerable catheter of claim 4, wherein one or more weld apertures are defined in the ring and the ring is welded to the distal tip at the one or more weld apertures, and wherein the ring is arranged on the distal tip such that the one or more weld apertures are angularly offset from the wire channel.

8. The steerable catheter of claim 1, wherein the annular body defines an overhang entrapment feature extending into the wire channel and thereby forming a gap sized to receive the wire.

9. The steerable catheter of claim 8, wherein the wire is secured to the distal tip by crimping the overhang entrapment feature onto the wire.

10. The steerable catheter of claim 8, wherein the wire is secured to the distal tip by welding at the overhang entrapment feature.

11. The steerable catheter of claim 8, further comprising a material deposited within the weld channel to encapsulate the wire.

12. The steerable catheter of claim 8, further comprising a sheath secured to the distal tip and arranged over top and circumferentially covering the weld channel.

13. A distal tip for a steerable catheter, comprising:
an annular body providing:
opposing first and second ends;
a first portion provided at the first end; and
a second portion contiguous with and extending proximally from the first portion; and
a wire channel defined in the second portion in the form of a path and sized to receive a wire such that opposing ends of the wire extend proximally from the annular body,
wherein a diameter of the first portion is larger than a diameter of the second portion such that a first shoulder is provided at an interface between the first and second portions.

14. The distal tip of claim 13, wherein the annular body further provides a third portion contiguous with and extending proximally from the second portion, and wherein the diameter of the second portion is larger than a diameter of the third portion such that a second shoulder is provided at an interface between the second and third portions.

15. The distal tip of claim 13, further comprising a ring mountable to the annular body at the second portion and at least partially covering the wire channel.

16. The distal tip of claim 15, wherein the ring comprises a discontinuous annular body having opposing first and second ends.

17. The distal tip of claim 15, wherein the ring is welded to the distal tip at one or more weld interfaces comprising butt welds applied at an interface between the ring and the first shoulder.

18. The distal tip of claim 15, wherein one or more weld apertures are defined in the ring and the ring is welded to the distal tip at the one or more weld apertures, and wherein the ring is arranged on the distal tip such that the one or more weld apertures are angularly offset from the wire channel.

19. The distal tip of claim 18, wherein a pocket is defined in the distal tip and the ring is arranged on the distal tip such that a corresponding one of the one or more weld apertures angularly aligns with the pocket.

20. The distal tip of claim 13, wherein the annular body defines an overhang entrapment feature extending into the wire channel and thereby forming a gap sized to receive the wire, and wherein the wire is secured to the distal tip by at least one of crimping the overhang entrapment feature onto the wire, and welding at the overhang entrapment feature.
